(19) United States
(12) Patent Application Publication
(10) Pub. No.: US 2006/0148903 A1
(43) Pub. Date: Jul. 6, 2006

(54) Capsaicinoid gel formulation and uses thereof

(75) Inventors: Ronald M. Burch, Wilton, CT (US); Timothy A. Anderson, Redding, CT (US); Jeff Lazar, Austin, TX (US)

Correspondence Address: DAVIDSON, DAVIDSON & KAPPEL, LLC, 485 SEVENTH AVENUE, 14TH FLOOR, NEW YORK, NY, 10018, US

(73) Assignee: AlgoRx Pharmaceuticals, Inc., Secaucus, NJ (US)

(21) Appl. No.: 11/286,059

(22) Filed: Nov. 23, 2005

Related U.S. Application Data
(60) Provisional application No. 60/630,577, filed on Nov. 24, 2004.

(51) Int. Cl. A61K 31/16; A61K 36/81

(52) U.S. Cl. 514/627; 424/760

(57) ABSTRACT

The present invention provides capsaicinoid gel formulations and methods for relieving pre- and post-surgical pain at a site in a human or animal by administering at a surgical site in a human or animal in need thereof a dose of capsaicinoid gel in an amount effective to attenuate post-surgical pain at the surgical site, the dose of capsaicin ranging from 100 μg to 10,000 μg.

This application claims the benefit of U.S. Provisional Patent Application No. 60/630,577, filed on Nov. 24, 2004, the disclosure of which is hereby incorporated by reference.

### FIELD OF THE INVENTION

The invention is directed to capsaicinoid formulations and methods for treating localized pain. In certain embodiments, the application is directed to a capsaicinoid gel formulation and its use intra-operatively for relieving post surgical pain in a variety of procedures.

### BACKGROUND OF THE INVENTION

Pain is an unpleasant sensation that occurs as a result of injury to the body or as a manifestation of a diseased state. Pain can be classified in many ways. For example, pain can be classified based on its duration (acute or chronic pain) and by the underlying cause (nociceptive or neuropathic).

Nociceptive pain results directly from local tissue injury whereas neuropathic pain follows nerve injury. Key features of nociceptive pain are that it can be experienced as sharp, dull, or aching, and that there may be radiation of the pain, or the perception of pain in a different area than where the nerves are being stimulated. For example, when a person experiences a heart attack, pain may radiate from the chest down the arms or up the neck, even though there is no tissue damage in these areas. Examples of nociceptive pain include pain from surgical incisions, bone pain from fractures or metastatic cancer, and pain from joint diseases such as osteoarthritis and rheumatoid arthritis.

Neuropathic pain occurs as a result of damage to, or dysfunction, of the nervous system. Neuropathic pain is frequently described as burning, tingling or having an electrical shock-like feeling. Another key feature of this type of pain is its paradoxical occurrence upon stimulation that otherwise would not be expected to cause pain. For example, a condition called trigeminal neuralgia may cause patients to feel extreme pain upon a light touch on the cheek. Examples of neuropathic pain include the pain resulting from diabetes and HIV infection, and postherpetic neuralgia, commonly called zoster, which is a painful condition caused by the chicken pox virus long after the initial infection has healed, in many cases years later. Neuropathic pain frequently coexists or follows nociceptive pain, as for example when a patient that has had a surgical procedure continues to experience pain long after the wound has healed.

Pain is a worldwide problem with serious health and economic consequences. The medical effort to treat pain, known as pain management, addresses a large and under-served market. According to IMS Health, the worldwide prescription market for pain drugs totaled over $23 billion in 2003, of which nearly $18 billion was spent in the United States. For example, in the United States medical economists estimate that pain results in approximately $100 billion of costs annually, as reported by the National Institutes of Health (NIH). Pain in the hospital is associated with increased length of stay, longer recovery times and poorer patient outcomes, all of which have health care quality and cost implications. Approximately 40 million Americans are unable to find relief from their pain, according to the NIH and more than 30 million Americans suffer chronic pain for which they visit a doctor.

Drugs are the principal means of treating pain. The pain management market is anticipated to grow at a compounded annual growth rate of 10% through 2010 due to a number of factors, including a rapidly aging population with an increasing need and desire to address pain-related ailments; longer survival times for patients with painful chronic conditions, such as cancer and AIDS; patients' increased demand for effective pain relief; and increasing recognition of the therapeutic and economic benefits of effective pain management by physicians, health care providers and payers.

Drugs that treat pain are referred to as analgesics. The type of analgesic selected for treatment depends upon the severity of the pain. For mild pain, the type of pain associated with many headaches or joint pain, weak analgesics such as acetaminophen or nonsteroidal anti-inflammatory drugs (NSAIDs) such as ibuprofen and Celebrex® (Pfizer) are used. For moderate pain, the type of pain associated with wisdom tooth extraction, other minor surgery and some arthritis pain, NSAIDs, weak opioids such as codeine or short-acting formulations of strong opioids such as Percocet® (Endo) may be used. Severe pain, which may occur following major surgery, advanced arthritis or cancer, requires strong opioids such as morphine, oxycodone, hydrocodone or fentanyl.

Despite widespread clinical use of drugs for pain, pain management remains less than optimal due to a variety of factors including: i) insufficient efficacy (NSAIDs are effective in treating only minor pain. Narcotics, the current standard of care for severe nociceptive pain, reduce pain less than 50% in most situations. Neuropathic pain is poorly treated by all existing analgesics); ii) side effects (NSAIDs often cause gastrointestinal ulcers, and more than 20,000 patients die each year from gastrointestinal bleeding induced by NSAIDs. One of the COX 2-selective NSAIDs, Vioxx® (Merck), has been shown to cause increased risk of heart attacks and possibly stroke. Use of narcotics is associated with nausea and vomiting in most patients. High-dose narcotics cause sedation and may also cause respiratory depression, or a decreased ability to breathe spontaneously. Narcotics used chronically can cause severe constipation that leads many patients to stop using them, and narcotics may sometimes cause severe itching. All of the drugs used to treat neuropathic pain frequently cause problems with coordination and sedation); iii) frequent dosing (Drugs used to treat neuropathic pain require frequent dosing that makes their use inconvenient, often leading to reduced patient compliance); iv) physical dependence (Narcotics, when used chronically, can cause physical dependence. Fear of physical dependence often influences clinicians to prescribe less than adequate doses of narcotic analgesics. Similar fears lead many patients to refuse narcotic analgesics); and v) diversion potential (Narcotics are often used by drug abusers, leading to considerable potential for diversion of legitimate narcotic analgesics for illicit uses. In fact, many pharmacies have removed high-dose narcotic analgesics from their inventories because of the risk of theft).

Pain management is of particular importance for treating severe post-surgical pain. There are over three million surgeries performed in the United States each year that result in severe post-surgical pain. Morphine and related narcotics, which are presently the standard of care for acute post-surgical pain, have serious side effects including respiratory depression, nausea, itching and sedation. In addition, many currently marketed drugs that treat pain require frequent dosing, which makes usage less convenient for patients.

As a result of the shortcomings of existing drugs that treat pain, capsaicin has become a front-runner of research and development for it use in treating pain.

Capsaicin, a pungent substance derived from the plants of the solanaceae family (hot chili peppers) has long been used as an experimental tool because of its selective action on the small diameter afferent nerve fibers C-fibers and A-delta fibers that are believed to signal pain. From studies in animals, capsaicin appears to trigger C-fiber membrane depolarization by opening cation channels permeable to calcium and sodium. Recently one of the receptors for capsaicin effects has been cloned. Capsaicin can be readily obtained by ethanol extraction of the fruit of capsicum frutescens or capsicum annum. Capsaicin is known by the chemical name N-(4-hydroxy-3-methoxybenzyl)-8-methylnon-trans-6-enamide. Capsaicin is practically insoluble in water, but freely soluble in alcohol, ether, benzene and chloroform. Therapeutically capsaicin has been used as a topical analgesic. Capsaicin is available commercially as Capsaicin USP from Steve Weiss & Co., 315 East 68^{th} Street, New York, N.Y. 10021 and can also be prepared synthetically by published methods. See Michalska et al., “Synthesis and Local Anesthetic Properties of N-substituted 3,4-Dimethoxyphenethylamine Derivatives”, Diss Pharm. Pharmacol., Vol. 24, (1972), pp. 17-25, (Chem. Abs. 77: 19271a), discloses N-pentyl and N-hexyl 3,4-dimethoxyphenylacetamides which are reduced to the respective secondary amines.

Capsaicin is listed in the pharmacopoeias of the United Kingdom, Australia, Belgium, Egypt, Germany, Hungary, Italy, Japan, Poland, Portugal, Spain, and Switzerland and has previously been listed in the United States Pharmacopoeia and the National Formulary. The FDA proposed monographs on analgesic drug products for over-the-counter (OTC) human use. These include capsaicin and capsicum preparations that are regarded as safe and effective for use as OTC external analgesics. Capsaicin is the only chemical entity of Capsicum recognized by the FDA. Capsaicin (USP) contains not less than 110% total capsaicinoids which typically corresponds to 63% pure capsaicin. USP capsaicin is trans-capsaicin (55-60%) and also contains the precursors dihydrocapsaicin and nordihydrocapsaicin.

Capsaicin mediated effects include: (i) activation of nociceptors in peripheral tissues; (ii) eventual desensitization of peripheral nociceptors to one or more stimulus modalities; (iii) cellular degeneration of sensitive A-delta and C-fiber afferents; (iv) activation of neuronal proteases; (v) blockage of axonal transport; and (vi) the decrease of the absolute number of nociceptive fibers without affecting the number of non-nociceptive fibers.

Capsaicin works to relieve pain by causing a localized degradation of the C neuron endings, and it is the only analgesic known to relieve pain by this mechanism. The activity of capsaicin results from its binding to, and activating, an ion channel called vanilloid receptor 1, or VR1. Under normal circumstances, when the VR1 ion channel is activated it opens for a short time, causing the C neurons to transmit a pain signal toward the brain. When capsaicin binds to, and activates VR1, it causes a series of events within the cell that degrade the pain-sensing endings, or terminals of the C neuron, thereby preventing the neuron from transmitting pain signals.

The effects of capsaicin are confined exclusively to the region of application because of low distribution to other areas of the body after capsaicin is administered. For example, after injection into a joint space or after application in a surgical procedure to the cut surfaces of skin, muscle and bone, capsaicin enters the blood slowly by diffusion from its site of initial application. Thereafter, capsaicin is highly metabolized, or broken down, by the liver into various inactive compounds, none of which retain any of the analgesic properties of capsaicin. As a consequence, capsaicin does not usually act at sites in the body distant from its initial application, nor is the body exposed to any derivatives of capsaicin that could act in a similar manner. By contrast, opioids and many other analgesics must be given by mouth or by intravenous injection, thereby subjecting the patient to circulation of high concentrations of drug. These high circulating concentrations may exert undesirable side effects by acting on parts of the body unrelated to pain perception. For example, opioids may cause constipation when used chronically. Opioids also may cause alteration of mood, and alertness, and can cause patients to feel drowsy, euphoric, or sleepy. These effects, when experienced by patients in the hospital, tend to increase rehabilitation time because patients are often sedated and therefore unable to begin the recovery process.

Humans have long been exposed to dietary sources of capsaicin-containing spices and to topical preparations used for a variety of medical indications. This vast experience has not revealed significant or lasting adverse effects of capsaicin exposure. The recent determination of potential therapeutic effects of capsaicin on unmyelinated sensory afferent nerve fibers require diligent consideration of this compound for further pharmaceutical development.

Because of the ability of capsaicin to desensitize nociceptors in peripheral tissues, its potential analgesic effects have also been assessed in various clinical trials. However, since the application of capsaicin itself frequently causes burning pain and hyperalgesia apart from the neuropathic pain being treated, patient compliance has been poor and the drop out rates during clinical trials have exceeded fifty percent. The spontaneous burning pain and hyperalgesia are believed to be due to intense activation and temporary sensitization of the peripheral nociceptors at the site of capsaicin application. This activation and sensitization occur prior to the desensitization phase. The activation phase could be a barrier to use of capsaicin because of the pain produced.

Prior publications describe topical administration of capsaicin for the treatment of various conditions. For example, U.S. Pat. No. 4,997,853 (Bernstein) describes methods and compositions utilizing capsaicin as an external analgesic. U.S. Pat. No. 5,063,060 (Bernstein) describes compositions and methods for treating painful, inflammatory or allergic disorders. U.S. Pat. No. 5,178,879 (Adekunle, et al.) describes methods for preparing a non-greasy capsaicin gel for topical administration for the treatment of pain. U.S. Pat. No. 5,296,225 (Adekunle, et al.) describes indirect methods of treating orofacial pain with topical capsaicin. U.S. Pat. No. 5,665,378 (Davis, et al.) describes transdermal therapeutic formulations comprising capsaicin, a nonsteroidal anti-inflammatory agent and pamabrom for the treatment of pain. U.S. Pat. No. 6,248,788 (Robbins, et al.) describes administration of 7.5% capsaicin cream in combination with marcaine epidural injections in patients suffering from long-term persistent foot pain. U.S. Pat. No. 6,239,180 (Robbins) describes combining capsaicin loaded patches with local anesthesia to treat peripheral neuropathy. The use of topical capsaicin has also been described in the art to treat conditions as diverse as post mastectomy pain syndrome (Watson and Evans, Pain 51: 375-79 (1992)); painful diabetic neuropathy (Tandan et al., Diabetes Care 15: 8-13 (1992)); The Capsaicin Study Group, Arch Intern Med 151: 2225-9 (1991); post-herpetic neuralgia (Watson et al., Pain 33: 333-40 (1988)), Watson et al., Clin. Ther. 15: 510-26 (1993); Bernstein et al., J. Am Acad Dermatol 21: 265-70 (1989) and pain in Guillian-Barre syndrome (Morganlander et al., Annals of Neurology 29:199 (1990)). Capsaicin has also been used in the treatment of osteoarthritis (Deal et al., Clin Ther 13: 383-95 (1991); McCarthy and McCarthy, J. Rheumatol 19: 604-7 (1992); Altman et al., Seminars in Arthritis and Rheumatism 23: 25-33 (1994).

Capsaicin is currently marketed for topical administration in the form of over-the-counter, low dose, non-sterile creams and patches, which tend to be poorly absorbed. There are more than thirty brands of creams and patches, including Capzasin-P® (Chattem) and Zostrix® (Rodlen Laboratories). These formulations are generally crude preparations of capsaicin that may contain other chemical entities. These over-the-counter preparations can be purchased widely without a prescription and are used topically by consumers to relieve pain in conditions such as osteoarthritis, shingles (herpes zoster), psoriasis and diabetic neuropathy.

It would therefore be advantageous to provide a topical capsaicinoid gel formulation and methods of use thereof that would be useful in different clinical settings as compared with current over-the-counter and prescription products. Specifically, it would be advantageous to provide a topical capsaicinoid gel formulation for use by physicians in the surgical setting prior to wound closure, e.g., in bunion removal surgery, hernia repair and other surgeries, by orthopedic surgeons and other physicians for the treatment of osteoarthritic knee joint disease and tendonitis, and for certain forms of localized neuropathic pain that are not amenable to treatment with currently marketed topical preparations.

### OBJECTS AND SUMMARY OF THE INVENTION

It is an object of the present invention to provide formulations and methods for providing pain relief in humans and animals by administering a dose of a topical capsaicinoid gel formulation to an open wound or surgical site for the treatment of acute or chronic pain, nociceptive and neuropathic pain.

It is an object of the present invention to provide formulations and methods for providing pain relief in humans and animals by administering a dose of a topical capsaicinoid gel formulation for the treatment of pre- and post-operative pain, cancer pain, pain associated with neurotransmitter dysregulation syndromes and orthopedic disorders, and/or localized severe or intractable pain.

It is an object of the present invention to provide formulations and methods for providing pain relief in humans and animals by administering intra-operatively a dose of a topical capsaicinoid gel formulation to a surgical site for the treatment of severe post-surgical pain.

It is another object of the present invention to provide formulations and methods for providing long-lasting analgesia without sedation in a human or animal.

It is a further object of the present invention to provide formulations and methods for alleviating the severe post-surgical pain suffered by patients following discharge from a clinical care facility.

It is another object of the present invention to provide formulations and methods for providing effective post-surgical analgesia such that the amount of narcotics taken by a patient or animal post-surgery would be reduced.

It is another object to provide formulations and methods for providing effective post-surgical analgesia thereby decreasing post-surgery rehabilitation time.

It is a further object of the invention to provide formulations and methods for treatment of sports-related injuries utilizing a topical capsaicinoid gel formulation.

It is a further object of the invention to provide formulations and methods for treatment of orthopedic disorders or injuries utilizing a topical capsaicinoid gel formulation.

It is a further object of the invention to provide formulations and methods for treating acute traumatic pain utilizing a topical capsaicinoid gel formulation.

It is a further object of the invention to provide formulations and methods for treating neuropathic pain utilizing a topical capsaicinoid gel formulation.

It is a further object of the invention to provide formulations and methods for treating nociceptive pain utilizing a topical capsaicinoid gel formulation.

It is a further object of the invention to provide formulations and methods for treating neurotransmitter-dysregulation syndromes utilizing a topical capsaicinoid gel formulation.

In accordance with the above objects and others, in certain embodiments of the present invention, there is provided a method for treating localized severe or intractable pain at a site in a human or animal in need thereof, comprising administering a dose of a topical capsaicinoid gel formulation at a discrete site in a human or animal in need thereof the dose of capsaicinoid being in an amount effective to attenuate or relieve pain at the site, preferably without eliciting an effect outside the site, and to attenuate pain emanating from the site, the dose ranging from about 100 μg to about 10,000 μg capsaicin or a therapeutically equivalent dose of a capsaicinoid other than capsaicin. In other words, the term “capsaicinoid” is meant to encompass formulations where the drug is capsaicin, e.g., natural or synthetic capsaicin, a capsaicinoid other than capsaicin, or a mixture of capsaicin with one or more other capsaicinoids (the total amount of all capsaicinoid drug being based on a therapeutically equivalent dose to dose from about 100 μg to about 10,000 μg capsaicin).

In certain other embodiments of the present invention, there is provided a method for treating post-surgical pain in a human or animal in need thereof, comprising administering intra-operatively a dose of a topical capsaicinoid gel formulation at a surgical site in a human or animal in need thereof, the dose of capsaicinoid being an amount effective to attenuate or relieve post-surgical pain at the surgical site, preferably without eliciting an effect outside the surgical site, and to attenuate or relieve pain emanating from the surgical site, the dose ranging from about 100 μg to about 10,000 μg capsaicin or a therapeutically equivalent dose of a capsaicinoid other than capsaicin.

In certain other embodiments, the dose of capsaicin may be greater than 10,000 μg. For example, the dose of capsaicin may be about 15,000 to about 50,000 μg.

In certain embodiments, the present invention is further directed to a gel formulation of a capsaicinoid, comprising from about 100 μg to about 10,000 μg of capsaicin or a therapeutically equivalent dose of a capsaicinoid other than capsaicin, a polysorbate base, a pharmaceutically acceptable gelling agent, and water for injection, the concentration of gelling agent in the water being sufficient to provide the gel formulation with a final viscosity from about 100 centipoises (cP) to about 50,000. In certain embodiments, the viscosity of the gel is in the range from about 100 to about 10,000 cP, preferably between 200 cP and 1,000 cP and more preferably between 250 cP to 350 cP with the most preferable viscosity in certain embodiments being approximately from about 300 to about 320 cP.

The present invention is further directed to a gel formulation of a capsaicinoid, comprising from about 100 μg to about 10,000 μg of capsaicin or a therapeutically equivalent dose of a capsaicinoid other than capsaicin, a polyalkylene glycol base, a pharmaceutically acceptable gelling agent, and water for injection, the concentration of gelling agent in the water being sufficient to provide the gel formulation with a final viscosity from about 100 cP (centipoises) to about 50,000 cP. In certain embodiments, the viscosity of the gel is in the range from about 100 to about 10,000 cP, preferably between 200 cP and 1,000 cP and more preferably between 250 cP to 350 cP with the most preferable viscosity in certain embodiments being approximately from about 300 to about 320 cP. Preferably, the gel formulation of the invention is not a liquid at room temperature (25 degrees C.).

In certain embodiments, the viscosity of the gel formulation is greater than 50,000 cP.

The viscosity of the gel formulations of the present invention can be measured by any means known in the art. For example, an LVDV-II+CP Cone Plate Viscometer and a Cone Spindle CPE-40 can be used to calculate the viscosity of the gel formulation of the present invention. The viscosity ranges referred to herein are measured at room temperature (25° C.).

In certain embodiments, the gel formulation may or may not include any alcohol.

In certain embodiments, the base may be any pharmaceutically acceptable solvent such as, but not limited to a polyalkylene glycol. In certain preferred embodiments, the polyalkylene glycol is a polyethylene glycol.

In certain other embodiments, the base may be any pharmaceutically acceptable surfactant such as, but not limited to, a polysorbate. In certain preferred embodiments, the polysorbate is polysorbate 80 (Tween 80).

In certain other embodiments, the gelling agent is one or more pharmaceutically acceptable cellulose, cellulose derivatives, or cellulose ethers (e.g., carboxymethylcellulose, ethylcellulose, hydroxyethylcellulose, hydroxymethylcellulose, hydroxypropylmethylcellulose, methylcellulose, and the like), one or more natural or synthetic gums (e.g., guar, xanthan, alginic acid and the like), or mixtures of any of the foregoing. In certain preferred embodiments, the gelling agent may be hydroxypropylmethyl cellulose (Methocel®).

In other embodiments, the gelling agent may be a pharmaceutically acceptable alginate, silicate or any combinations thereof.

In certain preferred embodiments of the present invention, the dose of capsaicinoid contained in a unit dose of the gel formulation is from about 100 μg to about 10,000 μg of capsaicin. In additional preferred embodiments, the dose of capsaicinoid contained in a unit dose of the gel formulation is from about 500 μg to about 5000 μg capsaicin, more preferably from about 1000 μg to about 3000 μg capsaicin, or a therapeutically equivalent amount of one or more capsaicinoids. Preferably, the capsaicinoid is administered in a pharmaceutically and physiologically acceptable vehicle for topical administration which may optionally further include one or more additional pharmaceutical excipients.

The dose of capsacinoid may be administered to the skin, a surgical incision site, a body cavity, a burn, or to a site of tissue injury. The gel formulation may be applied to exterior surfaces of skin or mucous membranes or to internal surfaces of muscles, organs, bones, and nerves that are accessible by surgery. The site of administration may be the skin, tissue, muscle or bones of the knee, elbow, hip, sternoclavicular, temporomandibular, carpal, tarsal, wrist, ankle, intervertebral disk, ligamentum flavum and any other bone and/or joint subject to pain.

The gel formulation of the invention may be administered to the desired site, e.g., via injection, infiltration, instillation, implantation, irrigation, or may be applied by painting, dropping, brushing, squirting or spraying. Administration by any of these methods may include the use of an application device such as, but not limited to, a syringe, a tube, a bottle (e.g., irrigation), a sterile pad (e.g., gauze); a dropper and the like.

In certain preferred embodiments, a local anesthetic may be administered prior to or concurrently with the dose of capsaicinoid in an amount and location effective to attenuate an initial hyperalgesic effect of the administered dose of capsaicinoid. The local anesthetic may be administered, e.g., by direct injection into the surgical site where the dose of capsaicinoid is administered, or as a proximal, regional, somatic, or neuraxial block. In other embodiments the local anesthetic may be administered topically to the surgical site. General anesthesia may be used, if necessary.

In certain embodiments, the administration of capsaicinoid at the discrete site provides pain attenuation or pain relief for at least about 48 hours to about 16 weeks.

In certain preferred embodiments, the capsaicinoid is capsaicin itself. In more preferred embodiments, the capsaicinoid comprises a purified or ultra-purified capsaicin.

In other embodiments, the capsaicinoid is a purified or ultra-purified trans-capsaicin. The ultra-purified capsaicin is at least about 97% trans-capsaicin, preferably about 98% trans-capsaicin and most preferably about 99% trans-capsaicin.

The single dose of topical capsaicinoid gel administered at a surgical site in accordance with the present invention is preferably in an amount effective to a) produce a selective, highly-localized destruction or incapacitation of C-fibers and/or A-delta fibers at the surgical site and/or in a localized area around the surgical site responsible for the initiation of pain for the purpose of reducing or eliminating pain arising from surgery, and b) minimize potential adverse consequences of C-fiber and/or A-delta activation and or damage outside of the locus of pain.

The present invention is also directed to a topical capsaicinoid gel formulation for attenuating post-surgical pain at and/or around a surgical site in a human or animal in need thereof, consisting essentially of from 10 μg to 10,000 μg of a capsaicinoid comprising trans-capsaicin and a pharmaceutically acceptable vehicle for topical application. In certain preferred embodiments, the dose of trans-capsaicin ranges from about 500 μg to about 5000 μg, more preferably from about 1000 μg to about 3000 μg.

In order that the invention described herein may be more fully understood, the following definitions are provided for the purposes of this disclosure:

The term “topical” shall mean administration of capsaicinoid gel to the skin, a surgical incision site, a body cavity, a burn, or to a site of tissue injury of a human or animal. The gel formulation may be applied to exterior surfaces of skin or mucous membranes or to internal surfaces of muscles, organs, bones, and nerves that are accessible by surgery.

As used herein, the term “capsaicinoid” means capsaicin, capsaicin USP, purified capsaicin, ultra-purified capsaicin, purified trans capsaicin, ultra-purified trans-capsaicin analogues and derivatives thereof (collectively referred to as capsaicinoids in this specification and appended claims) that act at the same pharmacologic sites, e.g., VR1, as capsaicin, unless otherwise specified.

The term “base” means any pharmaceutically acceptable agent capable of dissolving the capsaicinoid. For example, suitable bases may include, but are not limited to, any pharmaceutically acceptable solvent such as polyalkylene glycols or surfactant such as polysorbates.

Acute pain shall mean any pain that presents with a rapid onset followed by a short, severe course, e.g., post-surgical pain, headache, pain associated with cancer, fractures, strains, sprains, and dislocations of bones, joints, ligaments and tendons.

Chronic pain shall mean pain that lasts for a long period of time or is marked by frequent recurrence, e.g., pain associated with terminal illnesses, arthritis, autoimmune diseases; or neuropathic pain caused by degenerative diseases such as diabetes mellitus or spinal degeneration, or resulting from neural remodeling following traumatic injury or surgery.

As used herein, the term “local anesthetic” means any drug or mixture of drugs that provides local numbness and/or analgesia.

By co-administration it is meant either the administration of a single composition containing both the capsaicin and an additional therapeutically effective agent(s), e.g., local anesthetic or phenol, or the administration of a capsaicinoid and the additional therapeutically effective agent(s) as separate compositions within short enough time periods that the effective result is equivalent to that obtained when both compounds are administered as a single composition.

### DETAILED DESCRIPTION OF THE INVENTION

The invention will be described with reference to various specific and preferred embodiments and techniques, however, it should be understood that many variations and modifications can be made while remaining with the spirit and scope of the invention.

The formulations and methods disclosed herein can be used for treating pain at a surgical site with an effective amount of capsaicin or capsaicin analogue, hereinafter collectively referred to as “capsaicinoids”. In one preferred embodiment, the methods involve intra-operative administration of an effective amount of a topical capsaicinoid gel formulation to a surgical site in a human or animal for relieving post-surgical pain.

In another embodiment, the methods involve providing anesthesia to the surgical site where the capsaicinoid gel is to be administered, and then administering an effective amount of capsaicinoid gel to the surgical site to provide relief from post-surgical pain, e.g., for at least about 48 hours to about 16 weeks. The anesthesia can be provided topically, or parenterally directly to the site or at a remote site that causes anesthesia at the site where the capsaicinoid gel is to be administered. For example, epidural regional anesthesia can be provided to patients to which the capsaicinoid gel is to be administered at a surgical site located from the waist down. Alternatively, a local anesthetic may be administered as a regional block, a proximal block, a somatic block, or a neuraxial block. The anesthetic may be administered as a general anesthetic, as a spinal block, as an epidural block, or as a nerve block. Preferably, in the embodiments in which a local anesthetic is administered, the local anesthetic is administered prior to administration of the capsaicinoid gel, such that the local anesthetic has provided temporary anesthesia to the surgical site to be treated with the capsaicinoid gel.

Examples of local anesthetic agents which can be used include bupivacaine, ropivacaine, dibucaine, procaine, chloroprocaine, prilocalne, mepivacaine, etidocaine, tetracaine, lidocaine, and xylocalne, and mixtures thereof and any other art-known pharmaceutically acceptable local anesthetic. The local anesthetic can be in the form of a salt, for example, the hydrochloride, bromide, acetate, citrate, carbonate or sulfate. In certain embodiments, the local anesthetic agent is in the form of a free base. Preferred local anesthetic agents include, e.g., bupivacaine or lidocaine. For bupivacaine, the free base provides a slower initial release and avoids an early “dumping” of the local anesthetic at the site of administration. Other local anesthetics may act differently. Local anesthetic agents typically administered topically or parenterally may also be used in those cases where the means of administration results only in a local effect, rather than systemic.

The dose of local anesthetic will depend on the anesthetic being administered, the dosage form, e.g., topical or parenteral, as well as the site where the local anesthetic is administered. For example, in embodiments where the local anesthetic is administered via a regional block (e.g., an ankle block), the dose of anesthetic ranges from about 1 ml up to about 30 ml of a 0.5% solution (e.g., bupivacaine). In other embodiments a 3 mg/kg dose (maximum 200 mg) of a 2% solution (e.g., lidocaine) can be administered by intra-articular infiltration. In other embodiments the dose of local anesthetic can range between 0.5 ml to about 60 ml of a 0.25% to 5% solution. For topical application, the dose of the anesthetic may vary depending on the area being anesthetized, the vascularity of the tissues, the individual tolerance to anesthesia and the administration technique. For example, the maximum dose of an amide-type local anesthetic is about 25 mg. The maximum dose of an ester-type anesthetic is from about 50 mg to about 200 mg. The maximum does for other topically applied local anesthetics ranges from about 100 mg to about 200 mg.

In certain other embodiments, phenol can be administered at the site to be treated in place of (or in addition to) a local anesthetic to anesthetize the area. Phenol can preferably be administered prior to administration of the capsaicinoid gel, or can be co-administered with the dose of capsaicinoid gel. By co-administration it is meant either the administration of a single composition containing both the capsaicinoid gel and the phenol, or the administration of the capsaicinoid gel and the phenol as separate compositions within short enough time periods that the effective result is equivalent to that obtained when both compounds are administered as a single composition.

In the present invention, the capsaicinoid gel preferably contains capsaicin, purified capsaicin or ultra-purified capsaicin in natural or synthetic form. Administration of microgram quantities of a capsaicin, or a therapeutically equivalent dose of one or more capsaicinoids in a gel formulation, at a surgical site provides relief from post-surgical pain. A single dose from about 100 μg to 10,000 μg of capsaicin gel, or a therapeutically equivalent dose of one or more capsaicinoids in a gel formulation, is administered topically intra-operatively to produce a selective, highly-localized destruction or incapacitation of C-fiber and/or A-delta-fiber at the surgical site responsible for the initiation of pain for the purpose of eliminating pain arising from that locus, while minimizing potential adverse consequences of C-fiber and/or A-delta-fiber activation and/or damage outside of the locus of pain. In certain preferred embodiments, from about 500 to about 5000 micrograms of capsaicin gel, or a therapeutically equivalent dose of one or more other capsaicinoids in gel form, is administered at the surgical site. In certain preferred embodiments, the amount of capsaicin and/or preferably the range of capsaicin administered at the site is from about 1000 to about 3000 micrograms. In other words, the present invention is directed to topical administration of a single dose of capsaicinoid gel in an amount that is greatly reduced as compared to the dosage range previously considered useful by those skilled in the art to denervate the nerve fibers in a discrete, localized area without eliciting a systemic effect (e.g., an effect beyond that discrete, localized location).

Capsaicinoids (capsaicin analogues) with similar physiological properties, i.e., triggering C fiber membrane depolarization by opening of cation channels permeable to calcium and sodium, are known. For example, resiniferatoxin is described as a capsaicin analogue in U.S. Pat. No. 5,290,816 to Blumberg. U.S. Pat. No. 4,812,446 to Brand (Procter & Gamble Co.) describes other capsaicin analogues and methods for their preparation. U.S. Pat. No. 4,424,205 cites capsaicin analogues. Ton et al., Brit. J. Pharm. 10:175-182 (1955) discusses the pharmacological actions of capsaicin and its analogues. Capsaicin, capsaicin analogues and other capsaicinoids are also described in detail in WO 96/40079, the disclosure of which is hereby incorporated by reference. Capsaicinoids are also described in EP0 149 545, the disclosure of which is also hereby incorporated by reference.

Capsaicioids may be administered at the surgical site in replacement of, part of, or all of the dose of capsaicin, the capsaicinoid being administered in a therapeutically equivalent amount of capsaicin for which it is substituted. Where a capsaicinoid is selected to replace some or all of the capsaicin, the capsaicinoid can be selected from those compounds with similar physiological properties to capsaicin as are known in the art. Resiniferatoxin qualitatively resembles capsaicin in its activity, but differs quantitatively in potency (i.e. 103-104 fold more potent) and in relative spectrum of actions. For resiniferatoxin it is recommended to administer 0.1×10⁻³ to 5×10⁻² mg/kg, preferably 0.1×10⁻³ to 5×10⁻³ mg/kg, body weight of the subject for single application, or less upon multiple application. In certain embodiments, resiniferatoxin is administered in the range of 1×10⁻⁵ mg/kg to 5×10⁻² mg/kg to the subject. Resiniferatoxin also shows a somewhat different spectrum of action, providing greater relief of pain at a given dose. Therefore, the dose of resiniferatoxin should be at least 100 fold less than a dose of capsaicin alone.

Other suitable capsaicinoids for use in the present invention include, but are not limited to, N-vanillylnonanamides, N-vanillylsulfonamides, N-vanillylureas, N-vanillylcarbamates, N[(substituted phenyl)methyl]alkylamides, methylene substituted N[(substituted phenyl)methyl]alkanamides, N[(substituted phenyl) methyl]-cis-monosaturated alkenamides, N[(substituted phenyl)methyl]diunsaturated amides, 3-hydroxyacetanilide, hydroxyphenylacetamides, pseudocapsaicin, dihydrocapsaicin, nordihydrocapsaicin, homocapsaicin, homodihydrocapsaicin I, anandamide, piperine, zingerone, warburganal, polygodial, aframodial, cinnamodial, cinnamosmolide, cinnamolide, civamde, nonivamide, olvanil, N-oleyl-homovanillamidia, isovelleral, scalaradial, ancistrodial, β-acaridial, merulidial, scutigeral and any combinations or mixtures thereof.

In certain embodiments, the capsaicinoid utilized in the compositions and methods of the invention is capsaicin itself. In certain preferred embodiments, the capsaicin is in a purified or ultra-purified form obtained from the chemical purification of Capsaicin USP or chemical purification of synthetic capsaicin. In certain preferred embodiments, the purified capsaicin and/or ultra-purified capsaicin used in the gel formulations and methods of the present invention consists essentially of from about 95% to 99% of the trans-isomer. In certain preferred embodiments the ultra-purified capsaicin consists essentially of trans-capsaicin, e.g., having a purity of greater than about 97%, preferably greater than about 98%, more preferably greater than about 99% trans-capsaicin.

In contrast, Capsaicin USP contains only about 55-60% trans-capsaicin, with the remainder comprising the precursors dihydrocapsaicin and nordihydrocapsaicin.

The trans-isomer of capsaicin has its activity at the vanilloid receptor, thereby making the methods and formulations of the present invention especially useful for treating disorders or pain that can be alleviated through activation of the vanilloid receptors via the VR-1 mechanism.

The trans-isomer is preferably prepared in accordance with the method for synthesizing the trans-isomer of capsaicin from a four step process and purified as describe in Applicants U.S. patent application Ser. No. 10/821,473, filed Apr. 8, 2004, the disclosure of which is hereby incorporated by reference in its entirety. In accordance with U.S. patent application Ser. No. 10/821,473 said method for synthesizing the trans-isomer of capsaicin comprises a) alkylating 3-methyl butyne with halovaleric acid and/or haloalkanic acid to obtain 8-methyl-6-nonynoic acid and/or alkynoic acid analogues thereof; b) reducing said 8-methyl-6-nonynoic acid to obtain trans-8-methyl-nonenoic acid; c) activating the 8-methyl-nonenoic acid to obtain an acid chloride; and d) acylating 4-hydroxy-3-methoxybenzylamine hydrochloride with the acid chloride to obtain trans-capsaicin.

In certain embodiments, step a) of the method for preparation of the capsaicin for use in the present invention comprises the steps of: i) mixing anhydrous tetrahydrofuran (THF) with hexamethylphosphoramide (HMPA) and cooling the mixture to about −78° C. to about −75° C.; ii) adding to the mixture of step i) 3-methyl butyne followed by a dropwise addition of a base at a temperature from about −78° C. to about −65° C. to obtain a second mixture; iii) warming the second mixture up to about −30° C. and stirring for about 30 minutes; and iv) adding dropwise a solution of a halovaleric acid in anhydrous tetrahydrofuran at a temperature of about −30° C. for about 10 to about 15 minutes, then gradually warming to room temperature and stirring overnight to obtain a reaction mixture.

In certain other embodiments, there is provided a method for obtaining a crude step a) intermediate product further comprising the steps of: i) adding 3M hydrochloric acid (HCl) to a reaction mixture and extracting the reaction mixture with ethyl acetate; and ii) washing the extracted reaction mixture with brine to yield a crude product.

In certain embodiments, step b) of the method for preparation of the capsaicin for use in the present invention comprises the steps of: i) dissolving said 8-methyl-6-nonynoic acid in a mixture of anhydrous tetrahydrofuran and tertiary-butyl alcohol (t-BuOH) to obtain a solution and cooling the solution to about −55° C. to about −40° C.; ii) condensing ammonia (NH3) to the solution to a temperature of about −50° C. to about −40° C.; iii) adding sodium drips piece-wise and stirring from about 30 minutes to about 2 hours at a temperature from about −45° C. to about −30° C., and iv) adding ammonium chloride (NH4Cl), warming to room temperature and allowing the NH3 to evaporate overnight to obtain a reaction mixture. Step iii) of the step b) reaction may further comprise adding piece-wise lithium and stirring from about 30 minutes to about 2 hours at a temperature from about −65° C. to about −45° C.

In certain other embodiments crude step b) intermediate product further comprises the steps of: i) adding water to a reaction mixture; ii) acidifying the reaction mixture with 6N HCl to a pH of about 2 to about 3; iii) extracting the reaction mixture with ethyl acetate, washing with brine and drying over anhydrous sodium sulfate (Na2SO4); and iv) filtering and removing solvents under vacuum to obtain a crude step b) intermediate product.

In certain embodiments, step c) of the method for preparation of the capsaicin for use in the present invention comprises the steps of: i) adding dropwise a thionyl halide to the 8-methyl-nonenoic acid at room temperature for about 15 minutes to about 30 minutes to form a solution; ii) heating the solution at about 50° C. to about 75° C. for a period of about 1 hour; and iii) removing excess thionyl halide under vacuum at about 40° C. to about 45° C. to obtain a step c) intermediate product.

In certain embodiments, step d) of the method for preparation of the capsaicin for use in the present invention comprises the steps of: i) mixing 4-hydroxy-3-methoxy benzylamine hydrochloride and dimethylformamide (DMF); ii) adding portion-wise at room temperature to the mixture of step i) 5N sodium hydroxide (NaOH) and stirring for about 30 minutes; iii) adding acid halide in anhydrous ether dropwise at a temperature of about 0° C. to about 10° C. for about 20 minutes to about 1 hour; and, thereafter, iv) gradually warming the mixture to room temperature and stirring overnight. In certain embodiments step d) further comprises the steps of: i) adding water to the mixture and extracting the mixture with ethyl acetate to obtain an ethyl acetate extract; ii) washing said extract with 1N HCl and, thereafter, washing with sodium bicarbonate (NaHCO3); iii) washing the solution with brine and drying over anhydrous sodium sulfate (Na2SO4); and iv) filtering and removing solvents under vacuum to obtain a crude product.

In certain preferred embodiments, the method of preparing the trans-capsaicin or capsaicin intermediate after one or more of the steps (e.g., a), b), c) and/or d)) further comprises purifying the crude product by column chromatography, flash chromatography, or the like, using silica gel and eluting with a mixture of ethyl acetate/hexane to obtain a crude trans-capsaicin product.

After the capsaicin is formed via the four-step process as described above, the trans-capsaicin product is preferably subjected to purification process comprising the steps of: i) dissolving the crude trans-capsaicin product in a mixture of ether/hexane and heating the mixture to about 40° C. to about 45° C.; ii) cooling the mixture to room temperature while stirring for about 2 hours; and iii) filtering the mixture to provide a purified trans-capsaicin product.

In addition to the purification process(es) described above, the capsaicin is preferably subjected to a further purification process also referred to as a “semi-prep purification” or “semi-preparative purification” of capsaicin, which is also described in U.S. patent application Ser. No. 10/821,473, filed Apr. 8, 2004. In the semi-prep purification, natural capsaicin, synthetic capsaicin or previously purified natural or synthetic capsaicin is purified via the use of a semi-preparative HPLC (high performance liquid chromatography). When synthetic capsaicin undergoes the above-mentioned semi-preparative HPLC process, a trans-capsaicin product having a purity of greater than about 97%, preferably greater than about 98%, more preferably greater than about 99% capsaicin is provided.

In certain preferred embodiments, the active ingredient in the synthetic preparation comprises substantially pure trans-capsaicin (e.g. having no more than about 10% precursors or other capsaicin compounds such as cis-capsaicin). In more preferred embodiments, the preparation includes at least about 95% pure trans-capsaicin. In most preferred embodiments, the preparation includes at least about 99% ultra-pure trans-capsaicin. While the cis-isomer of capsaicin has activity via a number of mechanisms, VR-1 is not considered to comprise a major effect of this agent.

In view of the collective activity of the trans-isomer of capsaicin at the VR-1 receptor, it is contemplated that it is possible in certain embodiments of the present invention that the amount of trans-capsaicin included in the methods and formulations of the present invention will be reduced in comparison to a preparation which includes a less pure form of capsaicin (e.g., capsaicin USP).

In other embodiments of the present invention, the formulations and methods of the invention contemplate the use of a capsaicin agent consisting essentially of cis-capsaicin.

Administration of a single dose of topical capsaicinoid gel according to the methods of the present invention minimizes and/or prevents systemic delivery of the capsaicinoid for the purposes of: a) producing a selective, highly-localized destruction or incapacitation of C-fibers and/or A-delta fibers in a discrete, localized area responsible for the initiation of pain (e.g., trigger points, intra-articular spaces, bursa) for the purpose of reducing or eliminating pain arising from a discrete locus (i.e., producing antinociception), and b) minimizing potential adverse consequences of C-fiber and/or A-delta activation and or damage outside of the locus of pain (i.e., damage to homeostatic mechanisms, such as cardiac reflex [e.g., Bezold-Jarisch reflex] or micturation reflex [e.g., urge to void] or to nerve fibers in the central nervous system). The analgesic effect preferably provides pain relief for at least about 48 to about 120 hours, preferably from about 10 to about 21 days, more preferably from about 4 to about 5 weeks, even more preferably for at least about 6 to about 8 weeks, and most preferably for at least about 16 weeks or more.

The expected side effects of the dose of the capsaicinoid are believed to be from the intense nociceptor discharge occurring during the excitatory phase before nociceptor desensitization. However, the prior administration of an anesthetic, such as a nerve block, proximally or directly to the site of administration, eliminates or substantially reduces such side effects. If some “breakthrough pain” occurs despite the anesthetic, this pain may be treated by administering an analgesic such as a nonsteroidal anti-inflammatory agent or narcotic analgesic (i.e., the various alkaloids of opium, such as morphine, morphine salts, and morphine analogues such as normorphine). Administration of a single dose of the capsaicinoid gel formulation can be reapplied (repeated) to the skin at and/or around the surgical site, if necessary.

The topical gel formulations and methods of the present invention can be used for treating various conditions associated with pre- and post-surgical pain by providing pain relief at or around the surgical site. Painful conditions to be treated include, but are not limited to, nociceptive pain (pain transmitted across intact neuronal pathways), neuropathic pain (pain caused by damage to neural structures), pain from nerve injury (neuromas and neuromas in continuity), pain from neuralgia (pain originating from disease and/or inflammation of nerves), pain from myalgias (pain originating from disease and/or inflammation of muscle), pain associated with painful trigger points, pain from tumors in soft tissues, pain associated with neurotransmitter-dysregulation syndromes (disruptions in quantity/quality of neurotransmitter molecules associated with signal transmission in normal nerves) and pain associated with orthopedic disorders such as conditions of the foot, knee, hip, spine, shoulders, elbow, hand, head and neck.

The receptors involved in pain detection are aptly enough referred to as nociceptor-receptors for noxious stimuli. These nociceptors are free nerve endings that terminate just below the skin as to detect cutaneous pain. Nociceptors are also located in tendons and joints, for detection of somatic pain and in body organs to detect visceral pain. Pain receptors are very numerous in the skin, hence pain detection here is well defined and the source of pain can be easily localized. In tendons, joints, and body organs the pain receptors are fewer. The source of pain therefore is not readily localized. Apparently, the number of nociceptors also influences the duration of the pain felt. Cutaneous pain typically is of short duration, but may be reactivated upon new impacts, while somatic and visceral pain is of longer duration. It is important to note that almost all body tissue is equipped with nociceptors. As explained above, this is an important fact, as pain has primary warning functions, for example, impinging on the well-being of the patient and thereby causing the patient to seek medical assistance. Nociceptive pain includes, but is not limited to post-surgical pain, cluster headaches, dental pain, surgical pain, pain resulting from severe burns, post-partum pain, angina, genitor-urinary tract pain, pain associated with sports injuries (tendonitis, bursitis, etc . . . ) and pain associated with joint degeneration and cystitis.

Neuropathic pain generally involves abnormalities in the nerve itself, such as degeneration of the axon or sheath. For example, in certain neuropathies the cells of the myelin sheath and/or Schwann cells may be dysfunctional, degenerative and may die, while the axon remains unaffected. Alternatively, in certain neuropathies just the axon is disturbed, and in certain neuropathies the axons and cells of the myelin sheath and/or Schwann cells are involved. Neuropathies may also be distinguished by the process by which they occur and their location (e.g. arising in the spinal cord and extending outward or vice versa). Direct injury to the nerves as well as many systemic diseases can produce this condition including AIDS/HIV, Herpes Zoster, syphilis, diabetes, and various autoimmune diseases. Neuropathic pain is often described as burning, or shooting type of pain, or tingling or itching pain and may be unrelenting in its intensity and even more debilitating than the initial injury or the disease process that induced it.

Neuropathies treatable by the methods of the present invention include: syndromes of acute ascending motor paralysis with variable disturbance of sensory function; syndromes of subacute sensorimotor paralysis; syndromes of acquired forms of chronic sensorimotor polyneuropathy; syndromes of determined forms of genetic chronic polyneuropathy; syndromes of recurrent or relapsing polyneuropathy; and syndromes of mononeuropathy or multiple neuropathies (Adams and Victor, Principles of Neurology, 4th ed., McGraw-Hill Information Services Company, p. 1036, 1989). Syndromes of acute ascending motor paralysis are selected from the group consisting of acute idiopathic polyneuritis, Landry-Guillain-Barre Syndrome, acute immune-mediated polyneuritis, infectious mononucleosis polyneuritis, hepatitis polyneuritis; diptheric polyneuropathy; porphyric polyneuropathy; toxic polyneuropathy (e.g., thallium); acute axonal polyneuropathy; acute panautonomic neuropathy; vaccinogenic, serogenic, paraneoplastic, polyarteretic and lupus polyneuropathy.

Syndromes of subacute sensorimotor paralysis are selected from the group consisting of deficiency states (e.g., beriberi, pellagra, vitamin B12); heavy metal/industrial solvent poisonings (e.g., arsenic, lead); drug overdose (e.g., isoniazid, disulfuram, vincristine, taxol, chloramphenicol); uremic polyneuropathy; diabetes; sarcoidosis; ischemic neuropathy and peripheral vascular disease; AIDS; and radiation (radiotherapy). Syndromes of chronic sensorimotor are selected from the group consisting of carcinoma, myeloma and other malignancies; paraproteinemias; uremia; beriberi (usually subacute), diabetes, hypo/hyperthyroidism; connective tissue disease; amyloidosis; leprosy and sepsis. Genetic chronic polyneuropathies are selected from the group consisting of dominant mutilating sensory neuropathy (adult); recessive mutilating sensory neuropathy (childhood); congenital insensitivity to pain; spinocerebellar degenerations, Riley Day Syndrome; Universal Anesthesia Syndrome; polyneuropathies w/ metabolic disorder; and mixed sensorimotor-autonomic type polyneuropathies. Recurrent/relapsing polyneuropathy are selected from the group consisting of idiopathic polyneuritis; porphyria; chronic inflammatory polyradiculoneuropathy; mononeuritis multiplex; beriberi/drug overdose; refsum disease and tangier disease. Mono/multiple neuropathies are selected from the group consisting of pressure palsies; traumatic neuropathies (e.g., irradiation or electrical injury); serum, vaccinogenic (e.g., rabies, smallpox); herpes zoster; neoplastic infiltration; leprosy; diptheretic wound infections; migrant sensory neuropathy; shingles and post herpetic neuralgia.

Neurotransmitter-dysregulation pain syndromes, rather than involving abnormal or damaged nerves, result from normal nerves having disruptions in the quantity and/or quality of the various neurotransmitter molecules associated with signal transmission from one neuron to another. More specifically, sensory transmitters are released from the afferent nerve ending of one nerve cell and received by receptors at the afferent end of another nerve cell. They are chemical messengers which transmit the signal. There are numerous transmitters, including glutamate, serotonin, dopamine, norepinephrine, somatostatin, substance P, calcitonin gene-related peptide, cholecystokinin, opiates and saponins. Alterations in the quantity of transmitters and neuropeptide release, changes in the afferent receptor, changes of re-uptake of the transmitter and/or neuropeptides can all yield qualitative change of the neural signaling process. As a result, the aberrant signal transmission is interpreted by the body as pain. A representative neurotransmitter dysregulation syndrome that may be treated by the present invention includes fibromyalgia, which is a common condition characterized by a history of chronic generalized pain and physical exam evidence of at least 11 of 18 defined “tender point” sites in muscles and connective tissue (Wolfe et al., Arthritis Rheum 33:160-72, 1990). Commonly associated conditions include irritable bowel syndrome, headache, irritable bladder syndrome (interstitial cystitis), sleep disturbance, and fatigue (Goldenberg, Current Opinion in Rheumatology 8:113-123, 1996; Moldofsky et al., Psychosom Med 37:341-51, 1975; Wolfe et al., 1990; Wolfe et al., J Rheum 23:3, 1996; Yunus et al., Semin Arthritis Rheum 11: 151-71, 1981).

A predominant theory regarding the etiology of fibromyalgia holds that an imbalance and/or dysregulation of neurotransmitter function may occur within the central nervous system (CNS), either in the brain or spinal cord and in the relation of the CNS to muscle and connective tissue via regulatory nerve pathways (Goldenberg, 1996; Russell, Rheum Dis Clin NA 15:149-167, 1989; Russell et al., J Rheumatol 19:104-9, 1992; Vaeroy et al., Pain 32:21-6, 1988; Wolfe et al., 1996). Neurotransmitters are chemical messengers, amino acids, biogenic amines and neuropeptides, emitted from nerve cells that interact with receptors on other nerve cells, as well as other cell types, including muscle and immune cells. Neurotransmitter imbalance, which leads to increased pain experience, may include a qualitative and/or quantitative decrease in the function of such neurotransmitters as glutamate, serotonin, dopamine, norepinephrine, somatostatin, substance P, calcitonin gene-related peptide, cholecystokinin, opiates and saponins. Fibromyalgia is characterized by a relative deficit of serotonin effect and relative excess of substance P effect. This imbalance results in amplified modulation of pain-signaling in the central nervous system, resulting in neurogenic pain (Matucci-Cerinic, Rheumatic Disease Clinics of North America 19:975-991, 1993; Bonica, The Management of pain, Lea and Febiger, 2d ed., Philadelphia, pp. 95-121, 1990). Similar mechanisms may be at work to cause associated conditions; for example, dysregulation of neurotransmitter signaling in the bowel musculature, leading to irritable bowel syndrome symptoms such as cramping, diarrhea, and/or constipation.

Neurotransmitter-dysregulation pain syndromes include, but are not limited to the following: generalized syndromes, localized syndromes; craniofascial pain; vascular disease; rectal, perineum and external genitalia pain; and local syndromes of the leg/foot.

Generalized syndromes are selected from the group consisting of stump pain, causalgia, reflex sympathetic dystrophy, fibromyalgia or diffuse myofascial pain and burns. Localized syndromes are selected from the group consisting of trigeminal neuralgia; acute herpes zoster; panautonomic neuralgia; geniculate neuralgia (Romsay Hunt Syndrome); glossopharyngeal neuralgia; vagus nerve neuralgia and occipital neuralgia. Craniofacial pain includes temporomandibular pain. Suboccipital and cervical muskuloskeletal disorders are selected from the group consisting of myofascial syndrome, which includes cervical sprain cervical hyperextension (whiplash); stemocleidomastoid muscle; trapezius muscle; and stylohyoid process syndrome (Eagle's syndrome). Vascular disease is selected from the group consisting of Raynaud's disease; Raynaud's phenomenon; frosbite; erythema pernio (chilblains); acrocyanosis and livedo reticularis. Rectal, perineum and external genitalia pain are selected from the group consisting of iliohypogastric neuralgia; iliolinguinal nerve; genotifemoral nerve and testicular pain. Local syndromes of the leg/foot are selected from the group consisting of lateral cutaneous neuropathy (neuralgia paresthetica); oobturator neuralgia; femoral neuralgia; sciatica neuralgia; interdigital neuralgia of the foot (Morton's metatarsalgia or neurma); injection neuropathy and painful legs and moving toes.

Pain Intensity assessment scales are typically used by those of ordinary skill in the art to evaluate analgesic choices and therapeutic effects.

A Visual Analogue Scale (VAS) is a measurement instrument that measures a characteristic that is believed to range across a continuum of values and cannot easily be directly measured. For example, the amount of pain that a patient feels ranges across a continuum from none to an extreme amount of pain may be indirectly measured via the use of a VAS. Operationally a VAS is usually a horizontal line, 100 mm in length, anchored by word descriptors at each end, for example “no pain” at one end and “very severe pain” at the other end. The patient, marks on the line the point that they feel represents their perception of their current state. The VAS score is determined by measuring in millimeters from the left hand end of the line to the point that the patient marks. The 100-mm visual analog scale (VAS), a unidimensional scale that is versatile and easy to use, has been adopted in many settings.

The capsaicinoid gel formulations and methods described herein may be used to treat severe post-surgical pain wherein the capsaicinoid can be administered intra-operativley at the surgical site via application to the cut surfaces of the skin, muscle, and bone. The post-surgical pain may include but is not limited to acute or chronic pain associated with a surgical procedure, nociceptive and neuropathic pain, pre-operative pain, cancer pain, pain associated with neurotransmitter dysregulation syndromes and orthopedic disorders, sports-related injuries, acute traumatic pain, nociceptive pain, and neurotransmitter-dysregulation syndromes. For example, the gel formulations of the present invention can be used to treat post-surgical pain resulting from hernia repair, bunionectomy, mastectomy, hysterectomy, cholecystectomy, knee replacement surgery and other orthopedic surgery (e.g., back surgery). The above-mentioned surgical procedures are meant to provide examples of the types of surgical procedures that the gel formulations of the present invention may be useful. However, treatment of post-surgical pain associated with various other types of surgeries are contemplated.

Treatment of Chronic Post-Herniorrhaphy Pain

In a preferred embodiment, the capsaicinoid gel formulations and methods disclosed herein can be used for the treatment/attenuation of chronic post-herniorrhaphy pain. Chronic post-herniorrhaphy pain occurs in between 5-30% of patients, with social consequences limiting some type of activity in about 10% of patients and 1-4% of patients are referred to chronic pain clinics. Nerve damage is probably the most plausible pathogenic factor, but specific principles for therapy have not been evidence-based and range from usual analgesics to re-operation with mesh removal and various types of nerve sections without any demonstrated efficacy in sufficient follow-up studies with or without randomized data. In patients undergoing hernia repair, the dose of capsaicinoid gel can be administered intraoperatively to the surgical site where surgery is being performed or to the immediate area surrounding the incision. In other embodiments a subsequent dose of the capsaicinoid gel formulation can be administered to the site where the surgery was performed or to the immediate area surrounding the incision, if necessary.

Treatment of Post-Surgical Pain Following Hysterectomy

In another preferred embodiment, the capsaicinoid gel formulations and methods disclosed herein can be used for the treatment/attenuation of post-surgical pain following a hysterectomy. Hysterectomy is the second most common major surgery among women in the United States. Each year, more than 600,000 hysterectomies are done. About one third of women in the United States have had a hysterectomy by age 60. Hysterectomies can be performed by an incision in the abdomen (abdominal hysterectomy) or the vagina (vaginal hysterectomy). Abdominal hysterectomies are more common than vaginal hysterectomies and usually require a longer recovery time. In patients undergoing a hysterectomy, the dose of capsaicinoid gel can be administered intraoperatively to the surgical site where surgery is being performed (e.g., the abdominal tissue or vaginal region) or to the immediate area surrounding the incision. In other embodiments a subsequent dose of the capsaicinoid gel formulation can be administered to the site where the surgery was performed or to the immediate area surrounding the incision, if necessary.

Treatment of Post-Surgical Pain Following Bunionectomy

In another preferred embodiment, the capsaicinoid gel formulations and methods disclosed herein can be used for the treatment/attenuation of post-surgical pain following a bunionectomy. A bunion is a deformity that usually occurs at the head of the first of five long bones (the metatarsal bones) that extend from the arch and connect to the toes. The first metatarsal bone is the one that attaches to the big toe. The big toe is forced in toward the rest of the toes, causing the head of the first metatarsal bone to jut out and rub against the side of the shoe; the underlying tissue becomes inflamed and a painful bump forms. As this bony growth develops, the bunion is formed as the big toe is forced to grow at an increasing angle towards the rest of the toes. A bunion may also develop in the bone that joins the little toe to the foot (the fifth metatarsal bone), in which case it is known as a bunionette or tailor's bunion. Bunions often develop from wearing narrow, high-heeled shoes with pointed toes, which puts enormous pressure on the front of the foot and causes the foot and toes to rest at unnatural angles. Injury in the joint may also cause a bunion to develop over time. Genetics play a factor in 10% to 15% of all bunion problems; one inherited deformity, hallux valgus, causes the bone and joint of the big toe to shift and grow inward, so that the second toe crosses over it. Flat feet, gout, and arthritis increase the risk for bunions.

Surgical removal of a bunion is usually done while the patient is under general anesthesia (asleep and pain-free) and rarely requires a hospital stay. An incision is made along the bones of the big toe into the foot. The deformed joint and bones are repaired, and the bones are stabilized with a pin and/or cast. In patients undergoing bunion removal surgery, the dose of capsaicinoid gel can be administered intraoperatively to the surgical site where surgery is being performed (e.g., along the big toe) or to the immediate area surrounding the incision. In other embodiments a subsequent dose of the capsaicinoid gel formulation can be administered to the site where the surgery was performed or to the immediate area surrounding the incision, if necessary.

Treatment of Post-Surgical Pain Following Total Knee Replacement

In another preferred embodiment, the capsaicinoid gel formulations and methods disclosed herein can be used for the treatment/attenuation of post-surgical pain following a total knee replacement. Total knee replacement is a surgical procedure in which injured or damaged parts of the knee joint are replaced with artificial parts. The procedure is performed by separating the muscles and ligaments around the knee to expose the knee capsule (the tough, gristle-like tissue surrounding the knee joint). The capsule is opened, exposing the inside of the joint. The ends of the thigh bone (femur) and the shin bone (tibia) are removed and often the underside of the kneecap (patella) is removed. The artificial parts are cemented into place. The new knee will consist of a metal shell on the end of the femur, a metal and plastic trough on the tibia, and if needed, a plastic button in the kneecap. In patients undergoing knee replacement surgery, the dose of capsaicinoid gel can be administered intraoperatively to the surgical site where surgery is being performed (e.g., the knee capsule) or to the immediate area surrounding the incision. In other embodiments a subsequent dose of the capsaicinoid gel formulation can be administered to the site where the surgery was performed or to the immediate area surrounding the incision, if necessary.

Orthopedic Disorders

The capsaicinoid gel formulations and methods disclosed herein may be utilized to treat/attenuate pain associated with orthopedic disorders and post-surgical pain associated with orthopedic surgery. Pain associated with orthopedic disorders treatable via the use of the formulations and methods of the invention include but are not limited to disorders of the knee, shoulders, back, hip, spine, elbows, foot, hand and other disorders, which involve pain at a specific site, joint or body space. Orthopedic disorders affecting these locations include, but are not limited to bursitis, tendonitis, osteoarthritis, and rheumatoid arthritis. Bursitis is the inflammation of a bursa. Bursae are saclike cavities or potential cavities that contain synovial fluid located at tissue sites where friction occurs (e.g., where tendons or muscles pass over bony prominences). Bursae facilitate normal movement, minimize friction between moving parts, and may communicate with joints. In the normal state, the bursa provides a slippery surface that has almost no friction. A problem arises when a bursa becomes inflamed. The bursa loses its gliding capabilities, and becomes more and more irritated when it is moved. When the condition called bursitis occurs, the slippery bursa sac becomes swollen and inflamed. The added bulk of the swollen bursa causes more friction within already confined spaces. Also, the smooth gliding bursa becomes gritty and rough. Movement of an inflamed bursa are painful and irritating. Bursitis usually occurs in the shoulder (subacromial or subdeltoid bursitis). Other sites include the olecranon (miners' elbow), prepatellar (housemaid's knee) or suprapatellar, retrocalcaneal (Achilles), iliopectineal (iliopsoas) of the hip, ischial (tailor's or weaver's bottom) of the pelvis, greater trochanteric of the femur, and first metatarsal head (bunion). Bursitis may be caused by trauma, chronic overuse, inflammatory arthritis (e.g., gout, rheumatoid arthritis), or acute or chronic infection (e.g., pyogenic organisms, particularly Staphylococcus aureus; tuberculous organisms, which now rarely cause bursitis). Orthopedic disorders of the foot include, but are not limited to, heel spurs, corns, bunions, Morton's neuroma, hammertoes, ankle sprain, fractures of the ankle or metatarsals or sesamoid bone or toes, plantar fascitis and injuries to the achilles tendon. Orthopedic disorders of the hand include, but are not limited to, arthritis, carpal tunnel syndrome, ganglion cysts, and tendon problems such as lateral epicondylitis, medial epicondylitis, rotator cuff tendonitis, DeQuervian's tenosynovitis, and trigger finger/trigger thumb. Other orthopedic disorders include, but are not limited to, Paget's disease, scoliosis, soft-tissue injuries such as contusions, sprains and strains, long bone fractures and various other sports injuries some of which include patellar tendonitis and lumbar strain.

Treatment of non-infected acute bursitis has mainly consisted of temporary rest or immobilization and high-dose NSAIDs, sometimes narcotic analgesics, may be helpful. Voluntary movement should be increased as pain subsides. Pendulum exercises are particularly helpful for the shoulder joint. Aspiration and intrabursal injection of depot corticosteroids 0.5 to 1 ml (triamcinolone diacetate 25 or 40 mg/ml) mixed with at least 3 to 5 ml of local anesthetic after infiltration with 1% local anesthetic (e.g., lidocaine) is the treatment of choice when rest alone is inadequate. The depot corticosteroid dose and volume of mixture are gauged to the size of the bursa. Reaspiration and injection may be required with resistant inflammation. Systemic corticosteroids (prednisone 15 to 30 mg/day or equivalent for 3 days) are occasionally indicated in resistant acute cases after infection and gout have been excluded. Chronic bursitis is treated as acute bursitis, except that splinting and rest are less likely to be helpful. Surgery is rarely needed to treat bursitis and is usually done only in the chronic cases that have not improved with traditional therapy. The most common surgical treatment, if needed, is an Incision and Drainage (called an I and D) and is used only in cases of infected bursa. The surgeon first numbs the skin with an anesthetic and then opens the bursa with a scalpel. Finally, the surgeon drains the fluid present in the inflamed bursa. Sometimes it is necessary to excise the entire bursa surgically. This is indicated only if the bursal swelling causes problems.

The capsaicinoid gel formulations of the present invention may be administered topically at the surgical site. For example, in certain embodiments, the dose of capsaicinoid is administered directly to the cut surface of the skin, muscle and/or bone.

Tendonitis

The capsaicinoid gel formulations and methods disclosed herein may be utilized to treat/attenuate pain associated with tendonitis (inflammation of the tendons). When tendons become inflamed, the action of pulling the muscle becomes irritating and painful. The cause is often unknown. Most instances tendonitis occurs in middle-aged or older persons as the vascularity of tendons attenuates; repetitive microtrauma may increase injury. Repeated or extreme trauma (short of rupture), strain, or excessive (unaccustomed) exercise is most frequently implicated. The most common cause of tendonitis is overuse. Commonly, individuals begin an exercise program, or increase their level of exercise, and begin to experience symptoms of tendonitis. The tendon is unaccustomed to the new level of demand, and this overuse will cause an inflammation and tendonitis. Tendonitis produces pain, tenderness and stiffness near a joint which is aggravated by movement.

General practitioners commonly use non-steroidal anti-inflammatory drugs (NSAIDs) to treat tennis elbow, but there are no trials to date that have compared them with other painkillers and one study found no clinically important benefit over placebo. Symptomatic relief is provided by rest or immobilization (splint or cast) of the tendon, application of heat for chronic inflammation or cold for acute inflammation (whichever benefits the patient should be used), local analgesic drugs, and NSAIDs for 7 to 10 days. A critical review of the role of various anti-inflammatory medications in tendon disorders found limited evidence of short-term pain relief and no evidence of their effectiveness in providing even medium term clinical resolution. Use of corticosteroid injections provides mixed results in relief of pain and at times insufficient evidence to support their use. Injection of the tendon sheath with a depot corticosteroid (e.g., dexamethasone acetate, methylprednisolone acetate, hydrocortisone acetate) 0.5 to 1 mL mixed with an equal or double volume of 1% local anesthetic (e.g., lidocaine) has been utilized as a treatment, depending on severity and site. The injection is made blindly or proximal to the site of maximum tenderness if the specific inflammation site cannot be identified. Particular care should be taken not to inject the tendon per se (which offers greater resistance) because it may be weakened and rupture in active persons. Reexamination of a less inflamed site 3 or 4 days later often discloses the specific lesion, and a second injection can be made with greater precision. Rest of the injected part is advisable to diminish risk of tendon rupture. Although complications associated with intrarticular and soft tissue steroid injection are relatively uncommon, when a complication does occur, it can result in severe and disabling consequences for the subject. A small proportion of subjects fail to respond to only one injection of corticosteroid and some subjects who initially improve at four weeks had worst symptoms by six months. Therefore with this lack of consensus, no good evidence to support the use of local corticosteroid injections and the unknown long-term side-effects of using steroids, an alternative treatment must be sought.

In one embodiment of the present invention, pain associated with tendonitis of the knee, shoulders, hip, pelvis, spine, elbows, leg and foot is treated with a capsaicinoid injection undertaken in similar fashion as a localized corticosteroid injection. For example, in embodiments where the capsaicinoid gel formulation is used for the treatment/attenuation of pain associated with tendonitis or bursitis of the shoulder, the dose of capsaicinoid can be administered by application to the are skin surrounding the inflamed tendon.

Osteoarthritis

The capsaicinoid formulations and methods disclosed herein may be used to treat/attenuate pain associated with osteoarthritis and post-surgical pain associated with osteoarthritis surgery (degenerative joint disease). Osteoarthritis is characterized by the breakdown of the joint's cartilage. Cartilage is the part of the joint that cushions the ends of bones. Cartilage breakdown causes bones to rub against each other, causing pain and loss of movement. Most commonly affecting middle-aged and older people, osteoarthritis can range from very mild to very severe. It affects hands and weight-bearing joints such as knees, hips, feet and the back. There are many factors that can cause osteoarthritis, including but not limited to age, genetics, obesity, sports-related activities, work-related activities, or accidents. Treatment of osteoarthritis focuses on decreasing pain and improving joint movement, and may include: Exercises to keep joints flexible and improve muscle strength; Many different medications are used to control pain, including corticosteroids and NSAIDs, glucocorticoids injected into joints that are inflamed and not responsive to NSAIDS. For mild pain without inflammation, acetaminophen may be used; heat/cold therapy for temporary pain relief, joint protection to prevent strain or stress on painful joints; surgery (sometimes) to relieve chronic pain in damaged joints; and weight control to prevent extra stress on weight-bearing joints.

Post surgical pain associated with osteoarthritis may be treated/attenuated with the capsaicinoid gel formulations being applied to the cut surface of skin, muscle and/or bone at the surgical site, which surgical sites include but are not limited to disorders of the knee, shoulders, back, hip, spine, elbows, foot, hand and other disorders, which involve pain at a specific site, joint or body space.

Rheumatoid Arthritis

The capsaicinoid formulations and methods disclosed herein may be used to treat/attenuate pain associated with rheumatoid arthritis and post-surgical pain associated with arthritis surgery. Rheumatoid arthritis is a chronic, systemic, inflammatory disease that chiefly affects the synovial membranes of multiple joints in the body. Because the disease is systemic, there are many extra-articular features of the disease as well. Rheumatoid Arthritis can affect many joints in the body, including the knee, ankle, elbow, and wrist. Joints that are actively involved with the disease are usually tender, swollen, and likely demonstrate reduced motion. The disease is considered an autoimmune disease that is acquired and in which genetic factors appear to play a role. The capsaicinoid gel formulations may be administered topically by application to the cut surface of the skin, muscle or bone at the surgical site.

There are several different classes of drugs utilized to treat patients with the various types of rheumatic disease which maybe used in addition to the capsaicinoid treatment described herein, including analgesics to control pain, corticosteroids, uric acid-lowering drugs, immunosuppressive drugs, nonsteroidal anti-inflammatory drugs, and disease-modifying antirheumatic drugs.

Back Pain

The capsaicinoid gel formulations and methods disclosed herein may be used to treat/attenuate back pain and post-surgical pain associated with back surgery. Back pain is the second most common reason for doctor visits in the U.S. The causes of lower back pain are numerous. Some of the more common causes of lower back pain are: sudden injury to the back such as may occur in an auto accident, fall, sports, or other manner; gynecological conditions such as endometriosis, menstrual cramps, fibroid tumors, and pregnancy are sometimes the cause of lower back pain in women; and stress to the muscles, nerves, or ligaments in the lower back. Slipped discs, pinched nerves, sciatica, aging, and infections are other common causes of lower back pain.

The treatment of lumbar strain consists of resting the back (to avoid re-injury), medications to relieve pain and muscle spasm, local heat applications, massage, and eventual (after the acute episode resolves) reconditioning exercises to strengthen the low back and abdominal muscles.

Zgapophysial joints, better known as facet or “Z” joints, are located on the back (posterior) of the spine on each side of the vertebrae where it overlaps the neighboring vertebrae. The facet joints provide stability and give the spine the ability to bend and twist. They are made up of the two surfaces of the adjacent vertebrae, which are separated by a thin layer of cartilage. The joint is surrounded by a sac-like capsule and is filled with synovial fluid (a lubricating liquid that reduces the friction between the two bone surfaces when the spine moves and also nourishes the cartilage.) A problem (such as inflammation, irritation, swelling or arthritis) in the facet joint may cause low back pain. Diagnostic tests can show an abnormality in a facet joint, which may suggest that the facet joint is the source of the pain. However, sometimes normal study results can be present while the facet joint is still the source of pain, and abnormal results do not always implicate the facet joint.

To determine if a facet joint is truly the source of back pain, an injection of local anesthetic (.e.g., as a block) may be utilized. If an injection of a small amount of anesthetic or numbing medication into the facet joint reduces or removes the pain, it indicates that the facet joint may be the source of the pain. This is diagnostic use of the facet joint injection. Once a facet joint is pinpointed as a source of pain, therapeutic injections of anesthetic agents and anti-inflammatory medications may give pain relief for longer periods of time.

Facet joint injections are performed while the patient is awake, under a local anesthetic, and able to communicate. Sometimes, the health care provider may also administer drugs to make the patient more comfortable during the procedure. The injection is usually performed while the patient is lying on his or her stomach on an X-ray table. EKG, blood pressure cuffs and blood-oxygen monitoring devices may be hooked up prior to the injection process. Once the proper site has been determined, the physician will inject the anesthetic (often lidocaine or bupivacaine) and the anti-inflammatory (usually a corticosteroid.). This process may then be repeated depending on the number of affected facet joints. The capsaicinoid gel formulations may be administered in such situations to the area of skin at or near where the injection of local anesthetic or anti-inflammatory agent is to be administered thereby attenuating/preventing any post injection pain.

Heel Spur

The capsaicinoid gel formulations and methods disclosed herein may be used to treat/attenuate pain associated with heel spurs and post-surgical pain associated with heel spur surgery, which is a projection or growth of bone where certain muscles and soft tissue structures of the foot attach to the bottom of the heel. Most commonly, the plantar fascia, a broad, ligament-like structure extending from the heel bone to the base of the toes becomes inflamed, and symptoms of heel pain begin. As this inflammation continues over a period of time, with or without treatment, a heel spur is likely to form. If heel pain is treated early, conservative therapy is often successful and surgery is usually avoided. Early signs of heel pain are usually due to plantar fasciitis, the inflammation of the plantar fascia. It is probably the most common cause of heel pain seen by the podiatrist. It is seen in all groups of people; runners, athletes, week-end warriors, people who have jobs requiring a fair amount of standing, walking, or lifting, and those who have recently gained weight. Initially, patients receive taping of the foot and when indicated, cortisone injections or a short course an anti-inflammatory medication, taken orally. Exercises, night splints, and physical therapy are used as adjunct methods to try to reduce the inflammation. If successful, a custom made in shoe orthotic is made to control the abnormal stress and strain on the plantar fascia resulting in remission of the majority of the symptoms.

When capsaicinoid gel is used for the treatment of plantar fascia, the dose of capsaicinoid gel is preferably administered intra-operatively onto the cut surface of skin, muscle and/or onto the heel bone.

Laparoscopic Cholecystectomy

The capsaicinoid formulations and methods disclosed herein may be used to treat/attenuate post-surgical pain associated with laparoscopic cholecystectomy. Laparoscopic cholecystectomies have virtually replaced open surgical cholecystectomy. However, patients undergoing laparoscopic cholecystectomies still have pain. Pain control following surgery typically includes use of opioids, especially within the first several days after surgery. The administration of capsaicinoid gel in a patient who has undergone a laparoscopic cholecystectomy may reduce the amount of opioid consumption and postoperative pain scores associated with the procedure. In patients undergoing laparoscopic cholecystectomy, the dose of capsaicinoid gel can be administered directly to the cut surface of the skin, or to the tissue and/or muscle in the area of the incision or to the immediate area surrounding the surgical site.

The capsaicinoid gel formulations and methods disclosed herein may be used to treat/attenuate post-surgical pain associated with other laparoscopic surgical procedures, as well.

Dose of Gel Formulations

In preferred embodiments of the present invention, the dose of capsaicinoid gel contained in a unit dose is from about 100 μg to about 10,000 μg of capsaicin, preferably from about 500 μg to about 5000 μg capsaicin, more preferably from about 1000 μg to about 3000 μg capsaicin, or a therapeutically equivalent amount of one or more capsaicinoids.

In certain other embodiments, suitable doses of capsaicinoid gel for the treatment of nociceptive pain, neuropathic pain, pain from nerve injury, pain from myalgias, pain associated with painful trigger points, pain from tumors in soft tissues, pain associated with neurotransmitter-dysregulation syndromes and pain associated with orthopedic disorders range from about 1000 μg to about 10,000 jug of capsaicin (trans 8-methyl-N-vanillyl-6-noneamide), preferably from about 500 to about 500 micrograms, more preferably from about 1000 to 300 micrograms, with 1000 μg most preferred.

In certain preferred embodiments, an injection or topical dose of local anesthetic can be administered in proximity to the site prior to administration of the capsaicinoid gel, e.g., as described above and in the appended examples. In other embodiments, phenol can be used instead of or in addition to the local anesthetic.

Breakthrough Pain

The term “breakthrough pain” means pain which the patient experiences despite the fact that the patient is being or was administered generally effective amounts of, e.g., capsaicin. In conjunction with the use of the capsaicinoid formulations and methods described herein, it is contemplated that it is nonetheless possible that the patient will experience breakthrough pain. For the treatment of breakthrough pain, the individual may be further administered an effective amount of an analgesic in accordance with the treatment of pain in such situations performed by those skilled in the art. The analgesic may be any known to the person skilled in the art such as those selected from the group comprising gold compounds such as sodium aurothiomalate; non-steroidal anti-inflammatory drugs (NSAIDs) such as naproxen, diclofenac, flurbiprofen, ibuprofen ketoprofen, ketorolac, pharmaceutically acceptable salts thereof and the like; opioid analgesics such as codeine, dextropropoxyphene, dihydrocodeine, morphine, diamorphine, hydromorphone, hydrocodone, methadone, pethidine, oxycodone, levorphanol, fentanyl and alfentanil, para-aminophenol derivatives such as paracetamol, pharmaceutically acceptable salts thereof and the like; and salicylates such as aspirin.

Gel Formulations

Gels, sometimes referred to as jellies, have been defined various ways in the art. For example, the United States Pharmacopoeia defines gels as semisolid systems consisting of either suspensions made up of small inorganic particles or large organic molecules interpenetrated by a liquid. Gels can further consist of a single-phase or a two-phase system. A single-phase gel consist of organic macromolecules distributed uniformly throughout a liquid in such a manner that no apparent boundaries exist between the dispersed macromolecules and the liquid. Single-phase gels are usually prepared from synthetic macromolecules (e.g., carbomer) or from natural gums, (e.g., tragacanth). Single-phase gels are generally aqueous, but may also be made using alcohols and oils. Two-phase gels consist of a network of small discrete particles.

Gels can also be classified as being hydrophobic or hydrophilic. The bases of a hydrophobic gel usually consists of a liquid paraffin with polyethylene or fatty oils gelled with colloidal silica, or aluminum or zinc soaps. In contrast, the bases of hydrophobic gels usually consists of water, glycerol, or propylene glycol gelled with a suitable gelling agent (e.g., tragacanth, starch, cellulose derivatives, carboxyvinylpolymers, and magnesium-aluminum silicates).

Gels have been used to administer drugs topically or into a body cavity, e.g., nasal passage). However, unlike other topical gel formulations, the capsaicinoid gel formulations of the present invention may be administered intra-operatively to a surgical site, whereby the gel is applied directly to a cut surface of the skin or to the exposed tissue, muscle or bone at the surgical site. Accordingly, the gel formulations of the present invention must be suitable (e.g., sterile) for application to an open incision in order to reduce the risk of infection.

In order for the gel formulations of the present invention to be effectively applied to the surgical site, in certain embodiments the gel formulations preferably have a property whereby they are capable of being “painted on” to the surgical site. This “painted on” property can be obtained by providing a gel formulation having a specific viscosity measured in centipoises (cP). In certain embodiments of the invention, the viscosity of the gel is at least 100 centipoises (cP) to about 50,000. In certain embodiments, the viscosity of the gel is in the range from about 100 to about 10,000 cP, preferably between 200 cP and 1,000 cP and more preferably between 250 cP to 350 cP with the most preferable viscosity in certain embodiments being approximately from about 300 to about 320 cP.

In certain preferred embodiments, the viscosity of the gel formulation is greater than 50,000 centipoises (cP).

The capsaicinoid gel formulations can preferably be prepared by mixing the capsaicinoid together with a pharmaceutically and physiologically acceptable base to provide a capsaicinoid stock. The capsaicinoid stock of the present invention can be prepared by measuring the desired quantity of capsaicinoid required and placing the capsaicinoid into a glass beaker or vial. Next, the desired quantity of base may, due to the viscosity of the bases used and the difficulty of dispensing the desired quantity into a beaker by volume, be measured in weight. The desired quantity of base should then be slowly added to the beaker containing the capsaicinoid and gently stirred at room temperature for about 3 hours. The final capsaicinoid/base stock should then be filtered, e.g., through a 0.2 μm PES syringe filter, to sterilize.

In certain embodiments, the base may be sterilized using a filter prior to adding the capsaicin to form the stock solution. In other embodiments, the base may be sterilized using a gamma radiation prior to adding the capsaicin to form the stock solution.

In certain other embodiments, the capsaicinoid, the base, the capsaicinoid stock and/or the gelling agent and additional active ingredients may be sterilized using any other art known methods for sterilizing pharmaceutical products or ingredients.

Suitable bases for preparation of the capsaicinoid stock include, but are not limited to, any pharmaceutically acceptable solvent, surfactant or combinations thereof. For example, suitable solvents may include polyalkylene glycols such as, but not limited to, polyethylene glycol (PEG) and any combinations or mixtures thereof. Suitable surfactants include polysorbates such as, but not limited to, polysorbate 80 (Tween 80) and any combinations or mixtures thereof. In certain other embodiments, the base may be a combination of a pharmaceutically acceptable surfactant and solvent.

Other bases may include, sodium stearyl fumarate, diethanolamine cetyl sulfate, isostearate, polyethoxylated castor oil, benzalkonium chloride, nonoxyl 10, octoxynol 9, sodium lauryl sulfate, sorbitan esters (sorbitan monolaurate, sorbitan monooleate, sorbitan monopalmitate, sorbitan monostearate, sorbitan sesquioleate, sorbitan trioleate, sorbitan tristearate, sorbitan laurate, sorbitan oleate, sorbitan palmitate, sorbitan stearate, sorbitan dioleate, sorbitan sesqui-isostearate, sorbitan sesquistearate, sorbitan tri-isostearate), lecithin pharmaceutical acceptable salts thereof and combinations or mixtures thereof.

In certain preferred embodiments, the base may be polyethylene glycol. Polyethylene glycol is available in many different grades having varying molecular weights. For example, polyethylene glycol is available as PEG 200; PEG 300; PEG 400; PEG 540 (blend); PEG 600; PEG 900; PEG 1000; PEG 1450; PEG 1540; PEG 2000; PEG 3000; PEG 3350; PEG 4000; PEG 4600 and PEG 8000. For purposes of the present invention, all grades of polyethylene glycol are contemplated for use in preparation of the capsaicinoid stock.

In certain embodiments the polyethylene glycol used to prepare the capsaicinoid stock is preferably PEG 300.

In certain preferred embodiments, the base may be a polysorbate. Polysorbates are nonionic surfactants of sorbitan esters. Polysorbates useful in the present invention include, but are not limited to polysorbate 20, polysorbate 40, polysorbate 60, polysorbate 80 (Tween 80) and any combinations or mixtures thereof. In certain preferred embodiments, polysorbate 80 may be utilized as the pharmaceutically acceptable base.

After preparation of the capsaicinoid stock, the capsaicinoid stock can then be mixed together with a pharmaceutically and physiologically acceptable gelling agent(s) stock to provide for the capsaicinoid gel formulations of the present invention.

In certain embodiments, the gelling agent(s) stock can be prepared by weighing out the desired quantity of gelling agent and placing it into a glass beaker or vial. Next a desired quantity of water for injection is warmed and slowly added to the beaker containing the gelling agent while stirring for about 60 minutes. This mixture is then q.s. to a desired volume with preheated water for injection and stirred overnight.

In other embodiments, the gelling agent(s) may be added separately, and not part of a stock. For example, in certain embodiments, the gelling agent may be added to the capsaicin stock prior to or after the addition of additional ingredients, but before the addition of water. In other embodiments, the base and gelling agent may be combined prior to addition of the capsaicinoid.

Suitable gelling agents for use in preparation of the capsaicinoid gel formulation include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum and any combinations or mixtures thereof.

In certain preferred embodiments, hydroxypropylmethylcellulose (Methocel®) is utilized as the gelling agent.

Irregardless of the combination of base and gelling agents chosen, it is important that the viscosity of the gel formulation be within the desired range described above.

In addition to the above-mentioned bases and gelling agents, other pharmaceutically and physiologically acceptable excipients may be utilized in the gel formulations of the present invention. For example, viscosity increasing agents such as but not limited to bentonite, carbomer, ceratonia, cetostearyl alcohol, chitosan, colloidal silicon dioxide, cyclomethicone, hypromellose, magnesium aluminum silicate, maltitol, maltodextrin, medium chain triglycerides, polydextrose, polyvinyl alcohol, propylene glyceryl alginate, sodium alginate, tragacanth and any combinations or mixtures thereof.

In certain embodiments, the above-mentioned viscosity increasing agents may also be utilized as the gelling agent for the gel formulations herein.

In certain other embodiments, an additional surfactant (co-surfactant) and/or buffering agent can preferably be combined with one or more of the pharmaceutically acceptable vehicles previously described herein so that the surfactant and/or buffering agent maintains the product at an optimal pH for stability. The surfactant and/or buffering agent may also prevent the initial stinging or burning discomfort associated with capsaicinoid administration.

Suitable co-surfactants include, but are not limited to:

a) natural and synthetic lipophilic agents, e.g., phospholipids, cholesterol, and cholesterol fatty acid esters and derivatives thereof;

b) nonionic surfactants, which include for example, polyoxyethylene fatty alcohol esters, sorbitan fatty acid esters (Spans), polyoxyethylene sorbitan fatty acid esters (e.g., polyoxyethylene (20) sorbitan monooleate (Tween 80), polyoxyethylene (20) sorbitan monostearate (Tween 60), polyoxyethylene (20) sorbitan monolaurate (Tween 20) and other Tweens, sorbitan esters, glycerol esters, e.g., Myrj and glycerol triacetate (triacetin), polyethylene glycols, cetyl alcohol, cetostearyl alcohol, stearyl alcohol, polysorbate 80, poloxamers, poloxamines, polyoxyethylene castor oil derivatives (e.g., Cremophor® RH40, Cremphor A25, Cremphor A20, Cremophor® EL) and other Cremophors, sulfosuccinates, alkyl sulphates (SLS); PEG glyceryl fatty acid esters such as PEG-8 glyceryl caprylate/caprate (Labrasol), PEG-4 glyceryl caprylate/caprate (Labrafac Hydro WL 1219), PEG-32 glyceryl laurate (Gelucire 444/14), PEG-6 glyceryl mono oleate (Labrafil M 1944 CS), PEG-6 glyceryl linoleate (Labrafil M 2125 CS); propylene glycol mono- and di-fatty acid esters, such as propylene glycol laurate, propylene glycol caprylate/caprate; Brij® 700, ascorbyl-6-palmitate, stearylamine, sodium lauryl sulfate, polyoxethyleneglycerol triiricinoleate, and any combinations or mixtures thereof;

c) anionic surfactants include, but are not limited to, calcium carboxymethylcellulose, sodium carboxymethylcellulose, sodium sulfosuccinate, dioctyl, sodium alginate, alkyl polyoxyethylene sulfates, sodium lauryl sulfate, triethanolamine stearate, potassium laurate, bile salts, and any combinations or mixtures thereof;

d) cationic surfactants such as quarternary ammonium compounds, benzalkonium chloride, cetyltrimethylammonium bromide, and lauryldimethylbenzyl-ammonium chloride;

When one or more co-surfactants are utilized in the formulations of the invention, they may be combined, e.g., with a pharmaceutically acceptable vehicle and may be present in the final formulation, e.g., in an amount ranging from about 0.1% to about 20%, more preferably from about 0.5% to about 10%.

Suitable buffers include, but are not limited to acetate, bicarbonate, citrate, phosphate, pharmaceutically acceptable salts thereof and combinations or mixtures thereof. When one or more buffers are utilized in the formulations of the invention, they may be combined, e.g., with a pharmaceutically acceptable vehicle and may be present in the final formulation, e.g., in an amount ranging from about 0.1% to about 20%, more preferably from about 0.5% to about 10%. In certain embodiments of the present invention, the amount of buffer included in the gel formulations is preferably an amount such that the pH of the gel formulation does not interfere with the body's natural buffering system causing pain. Therefore, from about 5 mM to about 200 mM concentration of a buffer may be present in the gel formulation. In certain preferred embodiments, from about a 20 mM to about a 100 mM concentration of a buffer is present. Preferably, the concentration of buffer is such that a pH of the formulation is between 4 and 8, more preferably between 5 and 7. In certain preferred embodiments, the pH of the gel formulation is about 7.

In certain other embodiments, the gel formulation may be isotonic. Isotonic formulations may be provided by the addition of a tonicity agent. Suitable tonicity agents include, but are not limited to any pharmaceutically acceptable sugar, salt or any combinations or mixtures thereof, such as, but not limited to dextrose and sodium chloride. The tonicity agents may be present in an amount from about 100 mOsm/kg to about 500 mOsm/kg. In certain preferred embodiments, the tonicity agent is present in an amount from about 200 mOsm/kg to about 400 mOsm/kg and more preferably from about 280 mOsm/kg to about 320 mOsm/kg.

In certain embodiments, the capsaicinoid is capsaicin (natural or synthetic). The capsaicin utilized can be a purified or an ultra-purified form of natural capsaicin or synthetic capsaicin. Preferably the capsaicin is at least about 97%, more preferably 98% and most preferably 99% ultra-purified trans-capsaicin.

When capsaicin is utilized as the capsaicinoid, a desired amount of capsaicin is combined with the base to prepare a capsaicin stock. In certain embodiments, the concentration of capsaicin stock may range from about 0.1 mg/ml to about 5 mg/ml, preferably from about 1 mg/ml to about 2 mg/ml although various other concentrations of the capsaicin stock are contemplated depending on the solubility of capsaicin or capsaicinoid in the base.

The amount of base utilized in the gel formulations described herein will vary according to the concentration of capsaicinoid stock solution. In certain embodiments, the amount of base may range from about 1% to about 50%. In certain other embodiments, the amount of base may range from about 5% to 10%.

Once prepared, the stock solution may be mixed together with the gelling agent.

In certain embodiments, the capsaicin stock may be mixed together with at least about 50% gelling agent. In certain other embodiments, the capsaicin stock may be mixed together with from about 50% to about 99% gelling agent. In other embodiments, the capsaicin stock may be mixed together with from about 70% to about 80% gelling agent.

In certain other embodiments, when the capsaicin stock is prepared utilizing a polyalkylene glycol base, the stock is mixed together with from about 20% (v/v) to about 50% (v/v) gelling agent(s). In certain embodiments, the capsaicin/base stock is mixed together with from about 30% to 40% (v/v) gelling agent(s). Most preferably, the capsaicin/base stock is mixed together with 35% (v/v) gelling agent(s).

In other embodiments of the present invention, the capsaicin stock may be combined together with any additional ingredients mentioned above, prior to being combined with the gelling agent.

In certain preferred embodiments, the gel formulations of the present invention may or may not include alcohol.

In certain embodiments of the invention, the gel formulation may include an additional biologically active agent(s). Such biologically active agents include but are not limited to the following:

Anti-bacterial agents including, but not limited to penicillins, cephalosporins, vancomycin, bacitracin, cephalosporins, polymxyins, amikacin, doxycycline, nystatin, amphotericin-B, tetracyclines, chloramphenicol, erythromycin, neomycin, streptomycin, kanamycin, gentamicin, tobramycin, clindamycin, rifampin, nalidixic acid, flucytosine, griseofulvin, mixtures of any of the foregoing, and the like.

Antiviral agents including but not limited to vidarabine, acyclovir, ribavirin, amantadine hydrochloride, interferons, dideoxyuridine, mixtures of any of the foregoing and the like.

Antifungal agents including but not limited to nystatin, miconazole, tolnaftate, undecyclic acid and its salts, mixtures of the foregoing and the like.

Antiparasitic agents including but not limited to quinacrine, chloroquine, quinine, mixtures of the foregoing and the like.

Steroidal anti-inflammatory agents including but not limited to hydrocortisone, prednisone, fludrocortisone, triamcinolone, dexamethasone, betamethasone, mixtures of the foregoing and the like.

Antihistamines (H₂ antagonists) including, but not limited to diphenhydramine, chlorpheneramine, chlorcyclizine, promethazine, cimetidine, terfenadine, mixtures of the foregoing and the like.

Anesthetics including but not limited to cocaine, benzocaine, novocaine, bupivacaine, ropivacaine, dibucaine, procaine, chloroprocaine, prilocalne, mepivacaine, etidocaine, tetracaine, lidocaine, and xylocalne, phenol, mixtures of the foregoing and the like.

Suitable analgesic agents (including nonsteroidal anti-inflammatory agents) include, but are not limited to salicylic acid, salicylate esters and salts, acetaminophen, ibuprofen, morphine, phenylbutazone, indomethacin, sulindac, tolmetin, zomepirac, mixtures of the foregoing and the like.

Suitable antineoplastic agents include, but are not limited to methotrexate, 5-fluorouracil, bleomycin, tumor necrosis factors, tumor specific antibodies conjugated to toxins, mixtures of the foregoing and the like.

The additional (non-capsaicinoid) biologically-active agent(s) can be included in the compositions in the form of, for example, an uncharged molecule, a molecular complex, a salt, an ether, an ester, an amide, or other form to provide the effective biological or physiological activity.

The inclusion of an additional biologically active agent (in addition to capsaicinoid) will depend upon the condition to be treated, or surgical procedure undertaken.

The gel formulation of the present invention may alternatively or additionally contain preservatives to prevent microbial growth. Suitable preservatives for use in the present invention include, but are not limited to benzoic acid, boric acid, p-hydroxybenzoates, phenols, chlorinated phenolic compounds, alcohols, quarternary compounds, mercurials, mixtures of the foregoing and the like.

### DETAILED DESCRIPTION OF THE PREFERRED EMBODIMENTS

The following examples of gel formulations in accordance with the present invention are not to be construed as limiting the present invention in any manner and are only samples of the various formulations described herein.

### Example I

### Preparation of Capsaicin Stock

Preparation of 1 mg/ml Capsaicin Stock in PEG 300

21.0 grams of capsaicin (Lot# MCLS000826-3) was weighed into a 20 ml glass vial containing a 0.5 inch Flea micro stir bar. The addition of PEG 300 was performed by weight due to the high viscosity and difficulty of dispensing PEG 300 into the glass vial by volume. 23.62 grams of PEG 300 (density of 1.125 g/ml) was slowly dispensed in to the vial containing the solid bulk capsaicin and gently stirred at room temperature for 3 hours. A final visual assessment revealed a clear colorless homogenous solution with no particulate present. The capsaicin stock was subsequently filtered through a 0.2 μm PES syringe filter.

### Example II

### Preparation of Gelling Agents

A. 1% Sodium Carboxymethylcellulose Stock Solution

1.0 gram of sodium carboxymethylcellulose was weighed into a 100 ml beaker. 50 ml of water for injection (WFI) was warmed to 40° C. and dispensed slowly into the beaker containing the solid carboxymethylcellulose while stirring. The solution was stirred for 60 minutes. The solution was then q.s. to 100 ml with WFI preheated to 40° C. and stirred overnight. At the completion of the overnight stirring the solution appeared to have a homogenous thickness, clear consistency with a pale golden appearance, and no visible precipitates.

B. 1% Hydroxymethylcellulose Stock Solution

1.0 gram of hydroxymethylcellulose was weighed into a 100 ml beaker. 50 ml of water for injection (WFI) was warmed to 40° C. and dispensed slowly into the beaker containing the solid hydroxymethylcellulose while stirring. The solution was stirred for 60 minutes. The solution was then q.s. to 100 ml with WFI preheated to 40° C. and stirred overnight. The following morning the solution appeared to have a homogenous thickness and clear consistency with a pale golden appearance with no visible precipitates. This solution was slightly less viscous than the 1% sodium carboxymethylcellulose stock solution.

C. 0.5% Xanthan Gum Stock Solution

Xanthan gum forms a thick viscous solution when solubilized in water; therefore for this excipient a 0.5% stock solution was prepared. 1.0 gram of xanthan gum was weighed into a 250 ml beaker. 100 ml of WFI was warmed to 40° C. and dispensed slowly into the beaker containing the solid xanthan gum under stirring. The solution was stirred for 60 minutes then q.s. to 200 ml with WFI preheated to 40° C. and continued stirring overnight. It was noticed that the solubilization of xanthan gum was quite slow in comparison to the other gelling agents. At the completion of the overnight stirring the solution showed a homogenous thick consistency and had an opalescent white appearance. No visible precipitates were observed. This solution was slightly less viscous than the 1% sodium carboxymethylcellulose stock solution.

D. 1% Karaya Gum Stock Solution

1.0 gram of karaya gum was weighed into a 100 ml beaker. 50 ml of water for injection (WFI) was warmed to 40° C. and dispensed slowly into the beaker containing the solid karaya gum while stirring. The solution was stirred for 60 minutes. The solution was then q.s. to 200 ml with WFI preheated to 40° C. and stirred overnight. At the completion of the overnight stirring the solution showed a homogenous, slightly thick and clear consistency with a pale golden color with no visible precipitates or agglomeration. This solution is less viscous than the 1% sodium carboxymethylcellulose stock solution.

E. 5% Gum Arabic Stock Solution

5.0 grams of gum Arabic was weighed into a 100 ml beaker. 50 ml of water for injection was warmed at 40° C. and dispensed slowly into the beaker containing the solids of gum arabic while stirring. The solution was stirred for 60 minutes and subsequently q.s. to 100 ml with water for injection preheated to 40° C. and stirred overnight. At the completion of the overnight stirring the solution showed a homogenous, slightly thick and clear consistency with a pale golden color with no visible precipitates or agglomeration. This solution is less viscous than the 1% carboxymethylcellulose stock solutions.

F. 1% Alginic Acid Stock Solution

1.0 gram of algnic acid was weighed into a 100 ml beaker. 50 ml of water for injected was warmed at 40° C. and dispensed slowly into the beaker containing the solids of alginic acid while stirring. The solution was stirred for 60 minutes and subsequently q.s. to 100 ml with water for injection preheated to 40° C. and stirred overnight. At the completion of the overnight stirring the solution showed a homogenous, gelatinous thick and clear consistency with a pale golden color with no visible precipitates or agglomeration. This solution is slightly more viscous than the 1% carboxymethylcellulose stock solution.

Each of the stock solutions of gelling or thickening agents (A-F) were further diluted with water for injection to a final working solution as listed below in Tables I-III.

TABLE I
Gel Working Solutions: Sodium Carboxymethylcellulose,
Hydroxymethylcellulose, Karaya Gum and Alginic Acid

| % Gelling Agent | % Gelling Agent in |
|---|---|
| Stock Concentration | Working Solution |
| 1 | 1.0 |
| 1 | 0.9 |
| 1 | 0.8 |
| 1 | 0.7 |
| 1 | 0.6 |
| 1 | 0.5 |
| 1 | 0.4 |
| 1 | 0.3 |
| 1 | 0.2 |
| 1 | 0.1 |
| 1 | 0 |

TABLE II
Gel Working Solutions for Xanthan Gum

| % Gelling Agent | % Gelling Agent in |
|---|---|
| Stock Concentration | Working Solution |
| 0.5 | 0.5 |
| 0.5 | 0.45 |
| 0.5 | 0.4 |
| 0.5 | 0.35 |
| 0.5 | 0.3 |

TABLE III
Gel Working Solutions for Gum Arabic

| % Gelling Agent | % Gelling Agent in |
|---|---|
| Stock Concentration | Working Solution |
| 5 | 5.0 |
| 5 | 4.5 |
| 5 | 4.0 |
| 5 | 3.5 |
| 5 | 3.0 |

### Example III

### Preparation of Capsaicin Gel Formulation

Preparation of Capsaicin/PEG 300/Gelling Agent Formulation

A 1 mg/ml capsaicin stock formulated in PEG 300 was diluted at a ratio of 65:35 with the gelling agent working solutions prepared in Tables I-III above. Each of the excipient solutions were slowly blended by gentle swirl to the solubilized capsaicin stock. After combining all components and swirling for 10 minutes the solutions exhibited a non-homogenous appearance. All formulations were placed on a rotary rocker and mixed for 16 hours with gentle swirling. After 16 hours, all solutions exhibited a homogenous appearance.

The viscosity of each formulation (sample) was measured with a Brookfield LDDV-II+CP cone plate Viscometer (values expressed in centipoises (CP) units). An LV Series low viscosity Cone Spindle CPE-40 was used with a 0.5 ml sample volume. Tables IV-IX below describe the visual appearance and viscosity of the capsaicin/PEG 300/gelling agent formulations prepared. The final concentration of each gelling agent is also listed for each formulation. Note that regardless of formulation condition there was no visual precipitation of the capsaicin. As Tables IV-IX indicate, a variety of excipients and viscosities could be attained. Viscosities approaching that of K-Y®Brand Ultra Gel™ (˜310 CP) were achieved for example in formulations containing 0.175% sodium carboxymethylcellulose (Table IV).

TABLE IV
Capsaicin Formulations Containing Sodium Carboxymethylcellulose

| Final % Na CMC | Visual Appearance | Viscosity (CP) |
|---|---|---|
| — | REFERENCE K-Y ® BRAND | 310 |
| | ULTRA GEL ™ | |
| 0.350 | Thick clear very viscous gel, | >30,000 |
| | consistency of soft “Jell-O ®” | |
| 0.315 | Thick clear very viscous gel, | >30,000 |
| | consistency of soft “Jell-O ®” | |
| 0.280 | Thick clear very viscous gel, | >30,000 |
| | consistency of soft “Jell-O ®” | |
| 0.245 | Thick clear very viscous gel, | 2323 |
| | consistency of soft “Jell-O ®” | |
| 0.210 | Thick clear very viscous gel, | 694 |
| | consistency of runny “Jell-O ®” | |
| 0.175 | Clear Gel, consistency of K-Y ® | 273 |
| | BRAND ULTRA GEL ™ | |
| 0.140 | Clear gel, consistency <50% as thick | 177 |
| | as K-Y ® BRAND ULTRA GEL ™ | |
| 0.105 | Soft liquid | 79 |
| 0.070 | Soft liquid | 42 |
| 0.035 | Soft liquid | 22 |
| 0.000 | Soft liquid | 21 |

TABLE V
Capsaicin Formulations Containing Hydroxymethylcellulose

| Final % Na HMC | Visual Appearance | Viscosity (CP) |
|---|---|---|
| — | REFERENCE K-Y ® BRAND | 310 |
| | ULTRA GEL ™ | |
| 0.350 | Thick clear very viscous gel, | >30,000 |
| | consistency of soft “Jell-O ®” | |
| 0.315 | Thick clear very viscous gel, | >30,000 |
| | consistency of soft “Jell-O ®” | |
| 0.280 | Thick clear very viscous gel, | >30,000 |
| | consistency of soft “Jell-O ®” | |
| 0.245 | Thick clear very viscous gel, | >30,000 |
| | consistency of soft “Jell-O ®” | |
| 0.210 | Clear gel, consistency thicker than | 503 |
| | K-Y ® BRAND ULTRA GEL ™ | |
| 0.175 | Soft liquid gel, consistency <50% as | 162 |
| | thick as K-Y ® BRAND ULTRA | |
| | GEL ™ | |
| 0.140 | Soft liquid | 83 |
| 0.105 | Soft liquid | 60 |
| 0.070 | Soft liquid | 45 |
| 0.035 | Soft liquid | 22 |
| 0.000 | Soft liquid | 21 |

TABLE VI
Capsaicin Formulations Containing Xanthan Gum

| Final % Xanthan | | |
|---|---|---|
| Gum | Visual Appearance | Viscosity (CP) |
| — | REFERENCE K-Y ® BRAND | 310 |
| | ULTRA GEL ™ | |
| 0.175 | Clear pale golden, soft liquid | 38 |
| 0.158 | Clear pale golden, soft liquid | 30 |
| 0.140 | Clear pale golden, soft liquid | 33 |
| 0.123 | Clear pale golden, soft liquid | 25 |
| 0.105 | Clear pale golden, soft liquid | 30 |

TABLE VII
Capsaicin Formulations Containing Karaya Gum

| Final % Karaya | | |
|---|---|---|
| Gum | Visual Appearance | Viscosity (CP) |
| — | REFERENCE K-Y ® BRAND ULTRA | 310 |
| | GEL ™ | |
| 0.350 | Clear gel, consistency thicker than | 474 |
| | K-Y ® BRAND ULTRA GEL ™ | |
| 0.315 | Clear gel, consistency thicker than | 470 |
| | K-Y ® BRAND ULTRA GEL ™ | |
| 0.280 | Soft liquid gel, consistency <50% as | 205 |
| | thick as K-Y ® BRAND ULTRA | |
| | GEL ™ | |
| 0.245 | Soft liquid gel, consistency <50% as | 172 |
| | thick K-Y ® BRAND ULTRA | |
| | GEL ™ | |
| 0.210 | Soft liquid gel, consistency <50% as | 151 |
| | thick as K-Y ® BRAND ULTRA | |
| | GEL ™ | |

TABLE VIII
Capsaicin Formulations Containing Gum Arabic

| Final % Gum | | |
|---|---|---|
| Arabic | Visual Appearance | Viscosity (CP) |
| — | REFERENCE K-Y ® BRAND | 310 |
| | ULTRA GEL ™ | |
| 1.750 | Clear pale golden, soft liquid | 29 |
| 1.575 | Clear pale golden, soft liquid | 28 |
| 1.400 | Clear pale golden, soft liquid | 25 |
| 1.225 | Clear pale golden, soft liquid | 24 |
| 1.050 | Clear pale golden, soft liquid | 22 |

TABLE IX
Capsaicin Formulations Containing Alginic Acid Sodium Salt

| Final % AA | Visual Appearance | Viscosity (CP) |
|---|---|---|
| — | REFERENCE K-Y ® BRAND | 310 |
| | ULTRA GEL ™ | |
| 0.175 | Thick clear very viscous gel, | >30,000 |
| | consistency of soft “Jell-O” | |
| 0.140 | Clear gel, consistency thicker than | 591 |
| | K-Y ® BRAND ULTRA GEL ™ | |
| 0.105 | Soft liquid gel, consistency <50% as | 216 |
| | thick as K-Y ® BRAND ULTRA | |
| | GEL ™ | |
| 0.070 | Soft liquid gel, consistency <50% as | 181 |
| | thick as K-Y ® BRAND ULTRA | |
| | GEL ™ | |
| 0.035 | Clear, soft liquid | 21 |
| 0.000 | Clear, soft liquid | 21 |

### Example IV A-B

### Pre-Formulation Testing of Capsaicin

### Example IV A

### Two Component System

A known weight of capsaicin drug substance was weighed into a 4 ml Wheaton vial. A known volume of solvent was added and the sample placed in an ultrasonic bath for a minimum of 5 minutes. The temperature of the water bath was kept <25° C. at all times. Samples were transferred to a shaker bath at 25° C./60% RH and left for a minimum of 5 days. The samples were inspected and samples showing saturation (excess solid material remaining) removed for analysis. An additional known weight of capsaicin was added to the remaining samples and they were returned to the shaker bath. This was repeated until all samples had reached saturation.

Samples were filtered through a 0.45 μm PVDF Millipore Millex-HV hydrophilic disposable filter into a clean Wheaton vial. Sample absorbance was determined at 280 nm using 2 mm matched cuvettes. Samples were read against their equivalent solvent blank. Where necessary samples were diluted with methanol and these samples read against a methanol blank.

The concentration of each solution was determined by comparison with the absorbance of a capsaicin reference standard solution of known concentration.

Osmolality determinations were carried out by freezing point depression. The results are given in Table X.

TABLE X
Capsaicin Solubility in 2 Component Systems

| Sample Details (% w/v in | Capsaicin | Osmolality |
|---|---|---|
| Deionised Water) | Solubility (mg/ml) | (mOsm/kg) |
| 5% PEG 300 | 0.11 | 197 |
| 10% PEG 300 | 0.15 | — |
| 15% PEG 300 | 0.21 | — |
| 25% PEG 300 | 0.36 | — |
| 50% PEG 300 | 2.7 | — |
| 5% PEG 400 | 0.11 | 149 |
| 10% PEG 400 | 0.15 | — |
| 15% PEG 400 | 0.20 | — |
| 25% PEG 400 | 0.36 | — |
| 50% PEG 400 | 2.3 | — |
| 5% Propylene glycol | 0.09 | — |
| 10% Propylene glycol | 0.11 | — |
| 20% Propylene glycol | 0.16 | — |
| 50% Propylene glycol | 1.7 | — |
| 100% Propylene glycol | 160 | — |
| 0.5% Tween 80 | 0.53 | — |
| 1% Tween 80 | 1.2 | — |
| 2% Tween 80 | 2.2 | 3 |

### Example IV B

### Multi-Component System

Since PEG 300 and PEG 400 had showed similar solubility results on a % w/w basis, PEG 400 was selected for further work, as weight for weight it has a lower osmolality. Tween 80 results were very promising therefore the two materials were combined to investigate any possible synergistic effect on capsaicin solubility. Capsaicin is known to be soluble in ethanol therefore the effect of ethanol in combination with PEG 400 and Tween 80 was also investigated. The results are given in Table XI.

TABLE XI
Capsaicin Solubility in Multi Component Systems

| | Capsaicin | |
|---|---|---|
| Sample Details | Solubility | Osmolality |
| (% w/v in Deionised Water) | (mg/ml) | (mOsm/kg) |
| 0.5% Tween 80/6% PEG 400 | 1.1 | — |
| 0.5% Tween 80/6% PEG 400/5% ethanol | 1.2 | — |
| 0.5% Tween 80/6% PEG 400/10% ethanol | 1.4 | — |
| 0.5% Tween 80/9% PEG 400 | 1.0 | — |
| 0.5% Tween 80/12% PEG 400 | 1.1 | — |
| 1.25% Tween 80/6% PEG 400 | 1.8 | — |
| 1.25% Tween 80/9% PEG 400 | 1.8 | 334 |
| 1.25% Tween 80/12% PEG 400 | 1.7 | 501 |
| 2.0% Tween 80/6% PEG 400 | 2.2 | 204 |
| 2.0% Tween 80/9% PEG 400 | 2.0 | 347 |
| 2.0% Tween 80/12% PEG 400 | 2.6 | 514 |
| 6% PEG 400/5% ethanol | 0.09 | — |
| 6% PEG 400/10% ethanol | 0.38 | — |
| 0.5% Tween 80/5% ethanol | 1.1 | — |
| 1.25% Tween 80/5% ethanol | 2.5 | 1177 |

Tween 80 clearly has a significant impact on capsaicin solubility. The contribution from PEG 400, in comparison, is minor and there is no synergistic effect. Higher levels of Tween were therefore investigated. A formulation containing Tween 80 and capsaicin only would be hypotonic.

The solubility of capsaicin increases as the % Tween 80 increases in a linear manner over the range 0.5-2% Tween 80. For each 1% increase in Tween 80 the solubility increases by around 1 mg/ml. This might suggest that capsaicin is dissolved in the micelles of the surfactant, which would explain a proportionate solubility.

### Example V

The solubility of capsaicin was determined for the following vehicles by adding an excess of capsaicin and determining the saturation solubility by UV analysis of the supernatant.

TABLE XII
Sodium Chloride Gel Formulations

| Ingredients | pH 7.0 mg/ml | pH 5.5 mg/ml |
|---|---|---|
| Tween 80 | 20 | 20 |
| Citric acid, anhydrous | 3.84 | 3.84 |
| 1M NaOH | Qs pH 7.0 | Qs pH 5.5 |
| Sodium Chloride | Qs to 300 mOsm/kg | Qs to 300 mOsm/kg |
| Methocel K 100 | 12.5 | 12.5 |
| Water for Irrig./Inj. | to 1 ml | to 1 ml |

TABLE XIII
Glucose Gel Formulations

| Ingredients | pH 7.0 mg/ml | pH 5.5 mg/ml |
|---|---|---|
| Tween 80 | 20 | 20 |
| Citric acid, anhydrous | 3.84 | 3.84 |
| 1M NaOH | Qs pH 7.0 | Qs pH 5.5 |
| Sodium Chloride | Qs to 300 mOsm/kg | Qs to 300 mOsm/kg |
| Methocel K 100 | 12.5 | 12.5 |
| Water for Irrig./Inj. | to 1 ml | to 1 ml |

Method

Aliquots of 5 ml of each vehicle were dispensed into vials in duplicate and excess capsaicin added equivalent to 5 mg/ml. A small magnetic stirrer bar was introduced into vials containing the gel vehicle and mixed for approximately 5 minutes to disperse the capsaicin. All vials were sonicated for a total of 20 minutes maintaining the temperature below 25° C. The vials were stirred for a further hour.

One vial from each formulation was placed at 25° C./60% RH and one vial from each formulation was placed on a shaker at 2-8° C.

UV Analysis

Vials were removed from 25° C./60% RH after storage for 4 days. Vials stored at 2-8° C. were removed from the shaker after storage for 4 days and returned to 2-8° C. to equilibrate further before being removed after storage for a total of 7 days.

Preparation of Sample Solutions

A reference solution containing capsaicin 0.06 mg/ml in methanol was prepared. Samples were centrifuged for 20 minutes at 15000 rpm. The resultant supernatant from each vial was carefully transferred to fresh vials. The supernatant from each sample was diluted 1 in 50 with methanol and analyzed by UV at 280 nm using matched quartz cuvettes versus a methanol blank.

Results

The indicative solubilities for each formulation after storage at 2-8° C. and 25° C./60% RH are given in Tables XIV-XV.

TABLE XIV
Indicative solubility of capsaicin in Gel formulation after storage
at 2-8° C.

| | Concentration |
|---|---|
| Sample | mg/ml |
| gel pH 7.0 NaCl | 2.0 |
| gel pH 5.5 NaCl | 2.0 |
| gel pH 7.0 glucose | 2.0 |
| gel pH 5.5 glucose | 2.2 |

TABLE XV
Indicative solubility of capsaicin in Gel formulation after storage
at 25° C./60% RH

| | Concentration |
|---|---|
| Sample | mg/ml |
| gel pH 7.0 NaCl | 2.1 |
| gel pH 5.5 NaCl | 2.1 |
| gel pH 7.0 glucose | 1.9 |
| gel pH 5.5 glucose | 2.2 |

The solubility of capsaicin gel formulations after storage at 25° C./60% RH was as expected based on previous solubility work in 2% Tween 80 solutions, i.e. 2 mg/ml. There was no difference in solubility between formulations of different pH values with either glucose or sodium chloride present.

For samples stored at 2-8° C., supersaturation may have been reached during the sonication stage of the sample preparation. The vehicles were not chilled prior to addition of the capsaicin. The gel formulations stored at 2-8° C. have an unexpectedly higher concentration of 2 mg/ml similar to those formulations stored at 25° C./60% RH. This could be due to a number of factors:
The capsaicin was suspended evenly throughout the gel and was therefore in more intimate contact with the gel vehicle than with the RTU vehicle.
The capsaicin was better dispersed in the gel vehicle than in the RTU vehicle and did not settle or come out of solution within the time allowed as it was held in suspension by the gel structure of the vehicle.
The gel formulations were centrifuged at a much higher speed than the RTU formulations. This could have generated heat causing more capsaicin to go into solution. The gels were too viscous to be filtered and the higher speed was necessary to obtain a clear supernatant.

The final formulations for the vehicles after osmolality adjustment, as described above were as follows:

TABLE XVI
Sodium Chloride Gel Formulations

| Ingredients | pH 7.0 mg/ml | pH 5.5 mg/ml |
|---|---|---|
| Tween 80 | 20 | 20 |
| Citric acid, anhydrous | 3.84 | 3.84 |
| 1M NaOH | Qs pH 7.0 | Qs pH 5.5 |
| Sodium Chloride | 6.75 | 7.05 |
| Methocel K 100 | 12.5 | 12.5 |
| Water for Irrig./Inj. | to 1 ml | to 1 ml |

TABLE XVII
Glucose Gel Formulations

| | pH 7.0 | pH 5.5 |
|---|---|---|
| Ingredients | mg/ml | mg/ml |
| Tween 80 | 20 | 20 |
| Citric acid, anhydrous | 3.84 | 3.84 |
| 1M NaOH | Qs pH 7.0 | Qs pH 5.5 |
| Sodium Chloride | 37.60 | 39.15 |
| Methocel K 100 | 12.5 | 12.5 |
| Water for Irrig./Inj. | to 1 ml | to 1 ml |

Potential isotonic gel vehicles at pH 7.0 and 5.5 give rise to similar solubility results at 25° C. as simple solution in 2% Tween 80 (i.e., approximately 2 mg/ml capsaicin).

### Example VI A-D

### Preparation of Capsaicin Gel Formulation

### Example VI A

Preparation of Capsaicin/Tween 80/Gelling Agent Formulation (pH 7.0) with Sodium Chloride

Citric acid (3.84 mg or approx. 20 mM) and sodium chloride (qs to 300 mOsm/kg=6.75 mg) were dissolved in approximately ¾ volume of water (¾ ml). Tween 80 (20 mg) was added and stirred until dissolved. Capsaicin (2 mg) was added and stirred until dissolved. The pH of the mixture was adjusted to 7.0 with 1M sodium hydroxide. 12.5 mg of Hydroxypropylmethylcellulose (Methocel K 100M) was slowly added to the vortex while stirring vigorously until the hydroxypropylmethylcellulose was dissolved. Water was added to a final volume and the mixture was stirred until homogenous. The pH of the final mixture was then checked and adjusted to a pH of 7 by further addition of sodium hydroxide, when necessary. The viscosity of the gel was 10,500 cP.

TABLE XVIII
Capsaicin Gel Formulation (pH 7.0 with sodium chloride)

| Ingredients | mg/ml |
|---|---|
| Capsaicin | 2 |
| Tween 80 | 20 |
| Citric acid, anhydrous | 3.84 |
| 1M sodium hydroxide | Qs to pH 7.0 |
| Sodium chloride | 6.75 |
| Methocel K 100 | 12.5 |
| Water for Irrig./Inj. | to 1 ml |
| Osmolality (mOsm/kg) | 288 |

### Example VI B

Preparation of Capsaicin/Tween 80/Gelling Agent Formulation (pH 5.5) with Sodium Chloride

Citric acid (3.84 mg or approx. 20 mM) and sodium chloride (qs to 300 mOsm/kg=7.05 mg) were dissolved in approximately ¾ volume of water (¾ ml). Tween 80 (20 mg) was added and stirred until dissolved. Capsaicin (2 mg) was added and stirred until dissolved. The pH of the mixture was adjusted to 5.5 with 1M sodium hydroxide. 12.5 mg of Hydroxypropylmethylcellulose (Methocel K 100M) was slowly added to the vortex while stirring vigorously until the hydroxypropylmethylcellulose was dissolved. Water was added to a final volume and the mixture was stirred until homogenous. The pH of the final mixture was then checked and adjusted to a pH of 5.5 by further addition of sodium hydroxide, when necessary. The viscosity of the gel was 10,500 cP.

TABLE XIX
Capsaicin Gel Formulation (pH 5.5 with sodium chloride)

| Ingredients | mg/ml |
|---|---|
| Capsaicin | 2 |
| Tween 80 | 20 |
| Citric acid, anhydrous | 3.84 |
| 1M sodium hydroxide | Qs to pH 7.0 |
| Sodium chloride | 7.05 |
| Methocel K 100 | 12.5 |
| Water for Irrig./Inj. | To 1 ml |
| Osmolality (mOsm/kg) | 290 |

### Example VI C

Preparation of Capsaicin/Tween 80/Gelling Agent Formulation (pH 7.0) with Glucose

Citric acid (3.84 mg or approx. 20 mM) and glucose (qs to 300 mOsm/kg=37.60 mg) were dissolved in approximately ¾ volume of water (¾ ml). Tween 80 (20 mg) was added and stirred until dissolved. Capsaicin (2 mg) was added and stirred until dissolved. The pH of the mixture was adjusted to 7.0 with 1M sodium hydroxide. 12.5 mg of Hydroxypropylmethylcellulose (Methocel K 100M) was slowly added to the vortex while stirring vigorously until the hydroxypropylmethylcellulose was dissolved. Water was added to a final volume and the mixture was stirred until homogenous. The pH of the final mixture was then checked and adjusted to a pH of 7 by further addition of sodium hydroxide, when necessary. The viscosity of the gel was 10,500 cP.

TABLE XX
Capsaicin Gel Formulation (pH 7.0 with glucose)

| Ingredients | Mg/ml |
|---|---|
| Capsaicin | 2 |
| Tween 80 | 20 |
| Citric acid, anhydrous | 3.84 |
| 1M sodium hydroxide | Qs to pH 7.0 |
| Glucose anhydrous | 37.60 |
| Methocel K 100 | 12.5 |
| Water for Irrig./Inj. | to 1 ml |
| Osmolality (mOsm/kg) | 296 |

### Example VI D

Preparation of Capsaicin/Tween 80/Gelling Agent Formulation (pH 5.5) with Glucose

Citric acid (384 mg or approx. 20 mM) and glucose (qs to 300 mOsm/kg=39.15 mg) were dissolved in approximately ¾ volume of water (¾ ml). Tween 80 (20 mg) was added and stirred until dissolved. Capsaicin (2 mg) was added and stirred until dissolved. The pH of the mixture was adjusted to 5.5 with 1M sodium hydroxide. 12.5 mg of Hydroxypropylmethylcellulose (Methocel K 100M) was slowly added to the vortex while stirring vigorously until the hydroxypropylmethylcellulose was dissolved. Water was added to a final volume and the mixture was stirred until homogenous. The pH of the final mixture was then checked and adjusted to a pH of 5.5 by further addition of sodium hydroxide, when necessary. The viscosity of the gel was 10,500 cP.

TABLE XXI
Capsaicin Gel Formulation (pH 5.5 with glucose)

| Ingredients | mg/ml |
|---|---|
| Capsaicin | 2 |
| Tween 80 | 20 |
| Citric acid, anhydrous | 3.84 |
| 1M sodium hydroxide | Qs to pH 5.5 |
| Glucose anhydrous | 39.15 |
| Methocel K 100 | 12.5 |
| Water for Irrig./Inj. | to 1 ml |
| Osmolality (mOsm/kg) | 299 |

### Example VII

In this Example, the capsaicin gel formulations of Examples VI A-D are produced to a 100 ml scale.

Clinical Trials

Clinical trials may be performed to provide safety and efficacy data for the gel formulations of the present invention, with protocols similar to those set forth in the assignees co-pending U.S. patent application Ser. No. 10/742,621.

### CONCLUSION

It will be apparent to those skilled in the art that the capsacinoid gel formulations of the present invention may be utilized in many additional surgical and post-surgical treatments not specifically mentioned herein, and additionally it is contemplated that such formulations may be utilized at additional sites not specifically mentioned herein (including topically). Such obvious modifications are considered to be within the scope of the appended claims.

What is claimed is:

1. A method for treating post-surgical pain at a site in a human or animal comprising:
administering intra-operatively at a surgical site in a human or animal in need thereof a single dose of capsaicinoid gel in an amount effective to attenuate or relieve post-surgical pain at the surgical site without eliciting an effect outside the surgical site and to attenuate or relieve pain emanating from the surgical site, the dose ranging from about 100 μg to about 10,000 μg capsaicin or a therapeutically equivalent dose of a capsaicinoid other than capsaicin.

2. The method of claim 1, wherein said dose of capsaicin is from about 500 to about 5000 μg.

3. The method of claim 1, wherein said dose of capsaicin is from about 1000 to about 3000 μg.

4. The method of claim 1, wherein said dose of capsaicinoid is administered in a pharmaceutically and physiologically acceptable base for topical administration.

5. The method of claim 4, wherein said pharmaceutically acceptable base is a surfactant selected from the group consisting of a polysorbate and any combinations or mixtures thereof.

6. The method of claim 5, wherein said polysorbate is selected from the group consisting of polysorbate 20, polysorbate 40, polysorbate 60, polysorbate 80 and any combinations or mixtures thereof.

7. The method of claim 6, wherein said base is polysorbate 80.

8. The method of claim 5, wherein said dose of capsaicinoid is further administered in a pharmaceutically and physiologically acceptable gelling agent selected from the group consisting of carboxymethylcellulose, ethylcellulose, hydroxyethylcellulose, hydroxymethylcellulose, hydroxypropylmethylcellulose, hydroxypropylcellulose, methylcellulose, guar gum, karaya gum, xanthan gum, locust bean gum, alginic acid, starch, tragacanth, carboxyvinyl polymers and any combinations or mixtures thereof.

9. The method of claim 8, wherein said gelling agent is hydroxypropylmethylcellulose.

10. The method of claim 9, wherein said capsaicinoid is further administered in a pharmaceutically and physiologically acceptable excipient selected from the group consisting of a tonicity agent, a viscosity increasing agent, a co-surfactant, a buffering agent and any combinations or mixtures thereof.

11. The method of claim 10, wherein said tonicity agent is a pharmaceutically acceptable sugar or salt that is present in an amount from about 100 mOsm/kg to about 500 mOsm/kg.

12. The method of claim 10, wherein said tonicity agent is a pharmaceutically acceptable sugar or salt that is present in an amount from about 280 mOsm/kg to about 320 mOsm/kg.

13. The method of claim 10, wherein said tonicity agent is selected from the group consisting of dextrose, sodium chloride and any combinations or mixtures thereof.

14. The method of claim 10, wherein said viscosity increasing agent is selected from the group consisting of bentonite, carbomer, ceratonia, cetostearyl alcohol, chitosan, colloidal silicon dioxide, cyclomethicone, hypromellose, magnesium aluminum silicate, maltitol, maltodextrin, medium chain triglycerides, polydextrose, polyvinyl alcohol, propylene glyceryl alginate, sodium alginate, tragacanth and any combinations or mixtures thereof.

15. The method of claim 1, wherein said dose of capsaicinoid gel is administered intra-operatively to the cut surface of skin, tissue, muscle and bone at the surgical site.

16. The method of claim 1, further comprising co-administering a local anesthetic with said dose of capsaicinoid gel in an amount and location effective to attenuate an initial hyperalgesic effect of said administered dose of capsaicinoid.

17. The method of claim 16, wherein said local anesthetic is selected from the group consisting of dibucaine, bupivacaine, ropivacaine, etidocaine, tetracaine, procaine, chlorocaine, prilocalne, mepivacaine, lidocaine, xylocalne, 2-chloroprocaine, and acid addition salts or mixtures thereof.

18. The method of claim 16, wherein said local anesthetic is administered by direct injection into the site where said dose of capsaicinoid gel is administered.

19. The method of claim 16, wherein said local anesthetic is administered topically to the site where said dose of capsaicinoid gel is administered.

20. The method of claim 16, wherein said local anesthetic is administered to said site as a regional nerve block.

21. The method of claim 1, further comprising co-administering phenol with said dose of capsaicinoid gel in an amount and location effective to attenuate an initial hyperalgesic effect of said administered dose of capsaicinoid gel.

22. The method of claim 1, wherein said administration of capsaicinoid gel at said surgical site provides relief from post-surgical pain emanating from said site for at least about 48 hours up to about 16 weeks.

23. The method of claim 1, wherein said capsaicinoid comprises capsaicin.

24. The method of claim 1, wherein said capsaicinoid is other than capsaicin.

25. The method of claim 21, wherein said capsaicinoid is selected from the group consisting of resiniferatoxin, N-vanillylnonanamides, N-vanillylsulfonamides, N-vanillylureas, N-vanillylcarbamates, N[(substituted phenyl)methyl]alkylamides, methylene substituted N[(substituted phenyl)methyl]alkanamides, N[(substituted phenyl) methyl]-cis-monosaturated alkenamides, N[(substituted phenyl)methyl]diunsaturated amides, 3-hydroxyacetanilide, hydroxyphenylacetamides, pseudocapsaicin, dihydrocapsaicin, nordihydrocapsaicin anandamide, piperine, zingerone, warburganal, polygodial, aframodial, cinnamodial, cinnamosmolide, cinnamolide, isovelleral, scalaradial, ancistrodial, α-acaridial, merulidial, scutigeral, and any combinations or mixtures thereof.

26. The method of claim 25, wherein said capsaicinoid is resiniferatoxin.

27. The method of claim 23, wherein said capsaicin consists essentially of ultra-purified trans-capsaicin.

28. The method of claim 27, wherein said capsaicin consist essentially of 97% trans-capsaicin.

29. The method of claim 27, wherein said capsaicin consist essentially of 98% trans-capsaicin.

30. The method of claim 27, wherein said capsaicin consist essentially of 99% trans-capsaicin.

31. The method of claim 27, wherein said capsaicin is natural or synthetic capsaicin.

32. The method of claim 1, wherein said post-surgical pain is associated with median sternotomy, and the method further comprises administering to sternal edges of a human or animal undergoing a median sternotomy a single dose of capsaicinoid gel in an amount effective to denervate said sternal edges without eliciting an effect outside the sternal edge location, said dose of capsaicin gel ranging from about 11 g to about 3000 μg.

33. The method of claim 1, wherein said post-surgical pain is associated with chronic post-herniorrhapy, and the method further comprises administering to a site where hernia surgery was performed in a human or animal a single dose of a capsaicin gel in an amount effective to denervate said site, said dose of capsaicin gel ranging from about 500 μg to about 5000 μg.

34. The method of claim 1, wherein said post-surgical pain is associated with laparoscopic cholecystectomy, and the method further comprises administering to a site where said laparoscopic cholecystectomy was performed in a human or animal a single dose of a capsaicin gel in an amount effective to denervate said site, said dose of capsaicin gel ranging from about 500 μg to about 5000 μg.

35. The method of claim 1, wherein said post-surgical pain is associated with a bunionectomy, and the method further comprises administering into a wound opening resulting from a bunionectomy surgical procedure in a human or animal a single dose of a capsaicin gel in an amount effective to denervate said wound opening, said dose of capsaicin gel ranging from about 500 μg to about 5000 μg.

36. The method of claim 1, wherein said post-surgical pain is associated with a knee replacement, and, the method further comprises administering into a wound opening resulting from a knee replacement surgical procedure in a human or animal a single dose of a capsaicin gel in an amount effective to denervate said wound opening, said dose of capsaicin gel ranging from about 500 μg to about 5000 μg.

37. The method of claim 1, wherein said post-surgical pain is associated with a mastectomy, and the method further comprises administering into a wound opening resulting from a mastectomy surgical procedure in a human or animal a single dose of a capsaicin gel in an amount effective to denervate said wound opening, said dose of capsaicin gel ranging from about 500 μg to about 5000 μg.

38. The method of claim 1, wherein said dose of capsaicinoid is therapeutically equivalent to a dose of capsaicin in an amount from about 100 to about 10,000 μg.

39. The method of claim 1, wherein said dose of capsaicinoid is therapeutically equivalent to a dose of capsaicin in an amount from about 500 to about 5000 μg.

40. The method of claim 1, wherein said capsaicinoid comprises a mixture of capsaicinoids in a total amount equivalent to a capsaicin dose from about 100 μg to about 10,000 μg of capsaicin.

41. The method of claim 1, further comprising administering to said patient an analgesic to treat breakthrough pain.

42. The method of claim 1, further comprising co-administering with said capsaicinoid an additional biologically active agent selected from the group consisting of an anti-bacterial agent, antiviral agent, antifungal agent, antiparasitic agent, steroidal antiinflammatory agent, antihistamine, anesthetic, analgesic, antineoplastic and any combinations or mixtures thereof.

43. A topical gel formulation for attenuating or relieving post-surgical pain at a surgical site in a human or animal in need thereof, comprising a capsaicinoid selected from the group consisting of from 100 μg to 10,000 μg of capsaicin, a therapeutically equivalent amount of one or more other capsaicinoids, and combinations thereof, a pharmaceutically and physiologically acceptable base and a pharmaceutically and physiologically acceptable gelling agent.

44. The pharmaceutical formulation of claim 43, wherein said capsaicinoid comprises from about 500 μg to 5000 μg capsaicin.

45. The pharmaceutical formulation of claim 43, wherein said capsaicinoid comprises from about 1000 μg to 3000 μg capsaicin.

46. The pharmaceutical formulation of claim 45, wherein said capsaicin is at least about 97% trans-capsaicin.

47. The pharmaceutical formulation of claim 45, wherein said trans-capsaicin is at least about 98% trans-capsaicin.

48. The pharmaceutical formulation of claim 45, wherein said trans-capsaicin is at least about 99% trans-capsaicin.

49. The pharmaceutical formulation of claim 43, wherein said pharmaceutically acceptable base is a surfactant selected from the group consisting of a polysorbate and any combinations or mixtures thereof.

50. The pharmaceutical formulation of claim 49, wherein said polysorbate is selected from the group consisting of polysorbate 20, polysorbate 40, polysorbate 60, polysorbate 80 and any combinations or mixtures thereof.

51. The pharmaceutical formulation of claim 50, wherein said base is polysorbate 80.

52. The pharmaceutical formulation of claim 43, wherein said pharmaceutically and physiologically acceptable gelling agent is selected from the group consisting of carboxymethylcellulose, ethylcellulose, hydroxyethylcellulose, hydroxymethylcellulose, hydroxypropylmethylcellulose, hydroxypropylcellulose, methylcellulose, guar gum, karaya gum, xanthan gum, locust bean gum, alginic acid, starch, tragacanth, carboxyvinyl polymers and any combinations or mixtures thereof.

53. The pharmaceutical formulation of claim 52, wherein said gelling agent is hydroxypropylmethylcellulose.

54. The pharmaceutical formulation of claim 43, further comprising a pharmaceutically and physiologically acceptable excipient selected from the group consisting of a tonicity agent, a viscosity increasing agent, a surfactant, a buffering agent and any combinations or mixtures thereof.

55. The pharmaceutical formulation of claim 54, wherein said tonicity agent is a pharmaceutically acceptable sugar or salt that is present in an amount from about 100 mOsm/kg to about 500 mOsm/kg.

56. The pharmaceutical formulation of claim 54, wherein said tonicity agent is a pharmaceutically acceptable sugar or salt that is present in an amount from about 280 mOsm/kg to about 320 mOsm/kg.

57. The pharmaceutical formulation of claim 54, wherein said tonicity agent is selected from the group consisting of dextrose, sodium chloride and any combinations or mixtures thereof.

58. The pharmaceutical formulation of claim 54, wherein said viscosity increasing agents are selected from the groups consisting of bentonite, carbomer, ceratonia, cetostearyl alcohol, chitosan, colloidal silicon dioxide, cyclomethicone, hypromellose, magnesium aluminum silicate, maltitol, maltodextrin, medium chain triglycerides, polydextrose, polyvinyl alcohol, propylene glyceryl alginate, sodium alginate, tragacanth and any combinations or mixtures thereof.

59. The pharmaceutical formulation of claim 43, further comprising water for injection, wherein the concentration of gelling agent in said water being sufficient to provide said gel formulation with a final viscosity from about 100 cP to 50,000 cP.

60. The formulation of claim 59, wherein said viscosity is in the range from about 300 cP to about 320 cP.

61. The formulation of claim 59, wherein said viscosity is greater than 50,000 cP.

62. The formulation of claim 55, further comprising an additional biologically active agent selected from the group consisting of an anti-bacterial agent, antiviral agent, antifungal agent, antiparasitic agent, steroidal antiinflammatory agent, antihistamine, anesthetic, analgesic, antineoplastic and any combinations or mixtures thereof.

63. The gel formulation of claim 55, wherein said gel formulation further comprises a preservative selected from the group consisting of benzoic acid, boric acid, p-hydroxybenzoates, phenols, chlorinated phenolic compounds, alcohols, quarternary compounds, mercurials, and any mixtures of the foregoing.
